(19) United States
(12) Patent Application Publication
(10) Pub. No.: US 2019/0269662 A1
(43) Pub. Date: Sep. 5, 2019

(54) SOLID COMPOSITIONS OF ACTIVES, PROCESSES FOR PREPARING SAME AND USES OF SUCH SOLID COMPOSITIONS

(71) Applicant: The University of Liverpool, Liverpool (GB)

(72) Inventors: Steven Paul Rannard, Liverpool (GB); Andrew Owen, Liverpool (GB); Alison Savage, Liverpool (GB); Lee Tatham, Liverpool (GB)

(21) Appl. No.: 16/291,499

(22) Filed: Mar. 4, 2019

Related U.S. Application Data
(60) Provisional application No. 62/637,805, filed on Mar. 2, 2018.

(51) Int. Cl. A61K 31/439; A61K 47/32; A61K 9/16; A61K 47/20; A61K 9/107; A61P 31/14; A61K 9/00; A61K 9/10

(52) U.S. Cl. CPC A61K 31/439; A61K 47/32; A61K 9/1694; A61K 47/20; A61K 9/0053; A61P 31/14; A61K 9/0019; A61K 9/10; A61K 9/107

(57) ABSTRACT

The present invention provides a solid maraviroc composition, comprising nanoparticles including maraviroc dispersed within a mixture of at least one hydrophilic polymer and at least one surfactant; wherein the hydrophilic polymer is selected from polyvinyl alcohol (PVA), a polyvinyl alcohol-polyethylene glycol graft copolymer, polyethylene glycol, hydroxypropyl methyl cellulose (HPMC) and polyvinylpyrrolidone (PVP), or a combination thereof; and the surfactant is selected from a polyoxyethylene sorbitan fatty acid ester, sodium deoxycholate, dioctyl sodium sulfosuccinate and polyethyleneglycol-12-hydroxystearate, polyvinyl alcohol (PVA) or a combination thereof.

### RELATED APPLICATIONS

This application claims the benefit of priority to U.S. provisional application Ser. No. 62/637,805, filed Mar. 2, 2018, which is incorporated herein by reference in its entirety.

### INTRODUCTION

The present invention relates to improvements in compositions comprising one or more water-insoluble actives, processes for preparing such compositions and their uses.

There are a number of pharmaceutically active compounds which have limited solubility in water (water-insoluble actives). Improving the ease with which water-insoluble actives could be dispersed within aqueous solutions would also improve their pharmacokinetics. One approach is to formulate water-insoluble actives into solid drug nanoparticles (SDNs). However, such formulations still require further improvement in terms of the range of acceptable excipients and pharmacokinetic properties such as bioavailability, controlled release and tissue distribution.

An object of the invention is to provide improved methods of forming compositions comprising one or more water-insoluble actives.

A further object is to provide improved solid and aqueous compositions of water-insoluble actives.

### BACKGROUND TO ASPECTS OF THE INVENTION

Human Immunodeficiency Virus (HIV) is a major cause of morbidity and mortality in both the developed and the developing world. HIV is a retrovirus that causes acquired immunodeficiency syndrome (AIDS) in humans, which in turn allows life-threatening infections and cancers to thrive as the immune system progressively fails.

HIV infection typically occurs through the transfer of bodily fluids, such as blood, semen, vaginal fluid, pre-ejaculate, or breast milk, from one individual to another. HIV may be present within these bodily fluids as either the free virus, or as a virus present within infected immune cells. HIV-1 tends to be the most virulent form of HIV, and is transmitted as a single-stranded enveloped RNA virus which, upon entry into a target cell, is converted into double-stranded DNA by reverse transcription. This DNA may then become integrated into the host's DNA where it can reside in a latent from and avoid detection by the immune system. Alternatively, this DNA may be re-transcribed into RNA genomes and translated to form viral proteins that are released from cells as new virus particles, which can then spread further.

Maraviroc, sold commercially as Selzentry®, is a poorly water soluble anti-retroviral drug administered in the treatment of HIV. It is a CCR5 receptor antagonist, classed as an entry inhibitor as it acts to prevent HIV from gaining entry to macrophages and T-cells.

The structure of maraviroc is shown below:

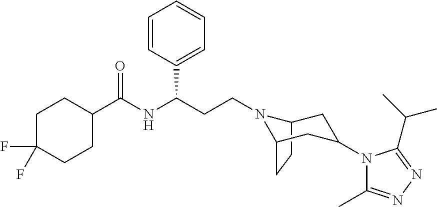 [SMILES: Cc1nnc(C(C)C)n1C1CC2CCC(C1)N2CC[C@H](NC(=O)C1CCC(F)(F)CC1)c1ccccc1]

Although maraviroc is effective in prolonging life expectancy in HIV sufferers, there are a number of drawbacks associated with the currently available formulations of maraviroc.

Maraviroc is poorly water-soluble and has a low oral bioavailability of approximately 33%, which is limited by poor permeability as well as affinity for CYP3A and several drug transporters. In addition, while once-daily doses are now the favoured option for HIV therapy, dose-limiting postural hypotension has been of concern when administering doses high enough to achieve this for maraviroc (particularly during coadministration of enzyme inhibitors). If these characteristics could be improved, the quantity of drug administered orally to patients may be reduced, limiting side effects whilst maintaining a therapeutically effective concentration.

What is more, suboptimal adherence to antiretroviral therapy, made more likely by frequent dosing regimens, can lead to insufficient drug exposure leading to viral rebound and increased likelihood of resistance. If the release rate of maraviroc were to be reduced, the duration that a therapeutically effective concentration could be maintained would be extended for a given dose of maraviroc.

There is also a need for dosage forms that permit the dosage to be easily varied on a patient-by-patient basis depending on factors such as the age (including paediatric dosing) and weight of the patient, as well as the severity and stage of the infection.

It is therefore an object of the present invention to provide improved formulations of maraviroc that address one or more of the drawbacks associated with the current maraviroc formulations.

In particular, it is an object of the invention is to provide maraviroc formulations exhibiting good cell penetration and a more optimum and effective distribution throughout the body.

Another object of the present invention is to provide maraviroc formulations with a high drug loading.

Another object of the present invention is to provide maraviroc formulations which require less frequent administration.

Another object of the present invention is to provide maraviroc formulations which permit lower overall dosage of maraviroc in HIV treatments.

### SUMMARY OF ASPECTS OF THE INVENTION

A first aspect of the present invention relates to a solid maraviroc composition, comprising nanoparticles comprising maraviroc dispersed within a mixture of at least one hydrophilic polymer and at least one surfactant;

wherein the hydrophilic polymer is selected from polyvinyl alcohol (PVA), a polyvinyl alcohol-polyethylene glycol graft copolymer, polyethylene glycol, hydroxypropyl methyl cellulose (HPMC) and polyvinylpyrrolidone (PVP), or a combination thereof; and

the surfactant is selected from a polyoxyethylene sorbitan fatty acid ester, sodium deoxycholate, dioctyl sodium sulfosuccinate and polyethyleneglycol-12-hydroxystearate, polyvinyl alcohol (PVA) or a combination thereof.

The nanoparticles comprising maraviroc comprising the solid maraviroc composition according to the first aspect of the present invention may have a Z-average particle diameter of less than or equal to 1 micron (μm).

The nanoparticles comprising maraviroc comprising the solid maraviroc composition according to the first aspect of the present invention may have a Z-average particle diameter between 100 and 800 nm.

The polydispersity of the nanoparticles comprising maraviroc comprising the solid maraviroc composition according to the first aspect of the present invention may be less than or equal to 0.8.

The hydrophilic polymer may be selected from from polyvinyl alcohol (PVA), a polyvinyl alcohol-polyethylene glycol graft copolymer and hydroxypropyl methyl cellulose (HPMC), or a combination thereof. Preferably the the hydrophilic polymer is PVA.

The surfactant may be selected from polysorbate 80, sodium deoxycholate, dioctyl sodium sulfosuccinate and polyethyleneglycol-12-hydroxystearate or a combination thereof. Preferably the surfactant is selected from polysorbate 80, sodium deoxycholate, dioctyl sodium sulfosuccinate, or a combination thereof.

In embodiments of the solid maraviroc composition according to the first aspect of the present invention the hydrophilic polymer is PVA and the surfactant is selected from polysorbate 80, sodium deoxycholate and dioctyl sodium sulfosuccinate.

The solid maraviroc composition according to the first aspect of the present invention may comprise:

60-80 wt % maraviroc;

15-25 wt % PVA; and

5-15 wt % dioctyl sodium sulfosuccinate.

Optionally the above composition may more specifically comprise at least one of 70 wt % maraviroc; 20 wt % PVA; and 10 wt % dioctyl sodium sulfosuccinate as an alternative to the expressed range of each component.

The solid maraviroc composition according to the first aspect of the present invention may comprise:

60-80 wt % maraviroc;

15-25 wt % PVA; and

5-15 wt % polysorbate 80.

Optionally the above composition may more specifically comprise at least one of 70 wt % maraviroc; 20 wt % PVA; and 10 wt % polysorbate 80 as an alternative to the expressed range of each component.

The solid maraviroc composition according to the first aspect of the present invention may comprise:

60-80 wt % maraviroc;

15-25 wt % PVA; and

5-15 wt % sodium deoxycholate.

Optionally the above composition may more specifically comprise at least one of 70 wt % maraviroc; 20 wt % PVA; and 10 wt % sodium deoxycholate as an alternative to the expressed range of each component.

A second aspect of the present invention relates to a pharmaceutical composition in a solid dosage form comprising a solid composition according to the first aspect of the present invention, and optionally one or more additional pharmaceutically acceptable excipients.

A third aspect of the present invention relates to a process for preparing a solid composition according to the first aspect of the present invention, the process comprising:

preparing an oil in water emulsion using a volatile oil comprising:
an oil phase comprising maraviroc; and
an aqueous phase comprising a hydrophilic polymer and a surfactant, each as defined in the first aspect of the present invention; and

removing the volatile oil and water to form the solid composition.

A fourth aspect of the present invention relates to a process for preparing a solid composition according to the first aspect of the present invention, the process comprising:

preparing a single phase solution comprising maraviroc, a hydrophilic polymer as defined in the first aspect of the present invention, and a surfactant as defined in the first aspect of the present invention, in one or more solvents; and

spray-drying the mixture to remove the one or more solvents to form the solid composition.

A fifth aspect of the present invention relates to an aqueous dispersion, comprising a plurality of nanoparticles dispersed in an aqueous medium, the nanoparticles comprising maraviroc, at least one hydrophilic polymer and at least one surfactant;

wherein the hydrophilic polymer is selected from polyvinyl alcohol (PVA), a polyvinyl alcohol-polyethylene glycol graft copolymer, polyethylene glycol, hydroxypropyl methyl cellulose and polyvinylpyrrolidone, or a combination thereof; and

wherein the surfactant is selected from a polyoxyethylene sorbitan fatty acid ester, sodium deoxycholate, dioctyl sodium sulfosuccinate, and polyethyleneglycol-12-hydroxystearate, polyvinyl alcohol (PVA) or a combination thereof.

The nanoparticles comprising maraviroc comprising the aqueous dispersion according to the fifth aspect of the present invention may have a Z-average particle diameter of less than or equal to 1 micron (μm).

The nanoparticles comprising maraviroc comprising the aqueous dispersion according to the fifth aspect of the present invention may have a Z-average particle diameter between 100 and 800 nm.

The average zeta potential of the nanoparticles comprising maraviroc comprising the aqueous dispersion according to the fifth aspect of the present invention, when dispersed in an aqueous medium may be between −100 and +100 mV.

A sixth aspect of the present invention relates to a process for preparing an aqueous dispersion according to the fifth aspect of the present invention, comprising dispersing a solid maraviroc composition according to the first aspect of the present invention in an aqueous medium.

A seventh aspect of the present invention relates to a pharmaceutical composition comprising an aqueous dispersion according to the fifth aspect of the present invention and optionally one or more additional pharmaceutically acceptable excipients.

An eighth aspect of the present invention relates to a solid composition according to the first aspect of the present invention, a pharmaceutical composition according to the second or seventh aspects of the present invention, or an aqueous dispersion according to the fifth aspect of the present invention, for use as a medicament.

A ninth aspect of the present invention relates to solid composition according to the first aspect of the present invention, a pharmaceutical composition according to the second or seventh aspects of the present invention, or an aqueous dispersion according to the fifth aspect of the present invention, for use in the treatment and/or prevention of retroviral infections (e.g. HIV).

A tenth aspect of the present invention relates to a method of treating and/or preventing a retroviral infection (e.g. HIV), the method comprising administering a therapeutically effective amount of a solid composition according to the first aspect of the present invention, a pharmaceutical composition according to the second or seventh aspects of the present invention, or an aqueous dispersion according to the fifth aspect of the present invention, to a patient suffering from or at risk of suffering from a retroviral infection.

An eleventh aspect of the present invention relates to a solid composition according to the first aspect of the present invention, a pharmaceutical composition according to the second or seventh aspects of the present invention, or an aqueous dispersion according to the fifth aspect of the present invention, such as HIV, wherein the solid composition, aqueous dispersion, or pharmaceutical composition is administered in combination with one or more other antiretroviral agents.

### DESCRIPTION OF THE DRAWINGS

FIG. 1A shows a 3-D bar chart representing the results from the study described in Example 1 herein, where “hits” are shown as grey bars and misses are shown as transparent bars. The Z-average particle diameter for each of the hits or misses is given on the vertical axis. FIG. 1A shows that maraviroc formulations with PVA and Tween20; PVA and Tween80; PVA and NDC; PVA and AOT; PVA and Solutol; Kollicoat and AOT; PEG 1000 and AOT; HPMC and AOT; and PVP K30 and AOT formed nanodispersions which met the nanodispersion quality assessment criteria.

FIG. 1B shows a 3-D bar chart representing the results from the study described in Example 2 herein wherein dispersions of compositions with increasing proportions of maraviroc were analysed by DLS. The Z-average particle diameter for each of the compositions is given on the vertical axis.

FIG. 2A shows apparent oral absorption (Pₐₚₚ ratio) and FIG. 2B apparent permeability (Pₐₚₚ cm s⁻¹) of a conventional [³H]-maraviroc preparation (<0.5% DMSO) and three [³H]-maraviroc nanodispersions across differentiated Caco-2 monolayers following 1 h incubation at 37° C., 5% CO₂. *, P<0.05; **, P<0.01 (unpaired, two-tailed t-test) (n=4).

FIG. 3 shows the Pₐₚₚ of [¹⁴C]-mannitol through Caco-2 following 1 h incubation at 37° C., 5% CO₂ to assess monolayer integrity post-incubation with conventional [³H]-MVC and [³H]-MVC nanosuspensions. Pₐₚₚ values <0.953×10⁻⁶ cm s⁻¹ considered to represent intact monolayer.

FIG. 4A shows the increased maraviroc plasma concentration achieved in vivo by dosing adult male Wister rats with [³H]-maraviroc at a concentration of 10 mg Kg⁻¹ as a conventional preparation (<5% DMSO) or as a maraviroc nanodispersion (^{MVC}SDN_{PVA/AOT}). The fragmented lines give the standard deviations of the mean for four rats in each group. FIG. 4B shows the tissue distribution of [³H]-MVC in adult male Wistar rats following oral dosing of [³H]-maraviroc at a concentration of 10 mg Kg⁻¹ as a conventional preparation (<5% DMSO) or as a maraviroc nanodispersion (^{MVC}SDN_{PVA/AOT}). Data are given as the mean±standard deviation for four rats in each group. **, P<0.01; ***, P<0.001 (unpaired, two-tailed t-test).

FIGS. 5A-5B show the [³H]-maraviroc release rate across a size selective membrane for both a conventional [³H]-maraviroc preparation (<5% DMSO) and the ^{MVC}SDN_{PVA/AOT} preparation using either transport buffer (FIG. 5A) or simulated interstitial fluid (FIG. 5B) as both donor and acceptor media in a RED assay. The average release rate constant was calculated over 6 h for each preparation and the error bars give the standard deviations of the mean from three replicates.

FIG. 6 shows the maraviroc exposure in adult male Wistar rats following a single intramuscular injection of [³H]-maraviroc (10 mg Kg⁻¹, 20 μCi mg [³H]-activity) in the biceps femoris either as a conventional preparation (<5% DMSO) or as the nanodispersion ^{MVC}SDN_{PVA/AOT}. Data is expressed as plasma concentrations of [³H]-maraviroc over the initial 24 h (insert) or until plasma concentrations fell below the limits of detection (<2 ng ml⁻¹). The fragmented lines give the standard deviations of the mean for three rats in each group.

FIG. 7 shows a 3-D bar chart displaying DLS data for the nanodispersions formed by dispersing maraviroc oil-blended SDN formulations (50 wt % maraviroc, 8.33 wt % Vitamin E) in water as per Example 7. “Hits” are shown as solid bars and near-misses are shown as transparent bars. The Z-average particle diameter for each of the hits or near-misses is given on the vertical axis. FIG. 7 shows that the maraviroc oil-blended SDNs with Vitamin E formed good nanodispersions when the combination of hydrophilic polymer and surfactant used was HPMC and Tween 80; HPMC and TPGS; or PVA and TPGS.

FIG. 8 shows a plot displaying the release of maraviroc from various compositions as measured by Rapid Equilibrium Dialysis (RED) over 6 hours as explained in Example 8. The compositions tested, in descending order of release rate, are: aqueous maraviroc (unformulated maraviroc); a conventional maraviroc SDN (ACS_14-70 wt % maraviroc; 20 wt % PVA; and 10 wt % AOT as described in 2); a maraviroc oil-blended SDN formulated with PVA and TPGS (PVA+TPGS); a maraviroc oil-blended SDN formulation with HPMC and TPGS (HPMC+TPGS); and a maraviroc oil-blended SDN formulation with HPMC and Tween 80 (HMPC+Tween 80).

FIG. 9 shows a bar chart expressing the quantity of maraviroc released over a 24 hour period as measured by RED for each of the maraviroc oil-blended SDNs expressed as a percentage of the total quantity of maraviroc in each formulation.

FIG. 10 shows a 3-D bar chart displaying DLS data for the nanodispersions formed by dispersing maraviroc oil-blended SDN formulations (50 wt % maraviroc, 8.33 wt % soybean oil) in water as per Example 9. “Hits” are shown as solid bars and near-misses are shown as transparent bars. The Z-average particle diameter for each of the hits or near-misses is given on the vertical axis. FIG. 10 shows that the maraviroc oil-blended SDNs with soybean oil formed good nanodispersions when the combination of hydrophilic polymer and surfactant used was HPMC and Tween 80; HPMC and TPGS; PVA and NDC; PVA and Tween 80; or PVA and TPGS.

FIG. 11 shows a 3-D bar chart displaying DLS data for the nanodispersions formed by dispersing maraviroc oil-blended SDN formulations (50 wt % maraviroc, 8.33 wt % soybean oil) in water as per Example 10. “Hits” are shown as solid bars and near-misses are shown as transparent bars. The Z-average particle diameter for each of the hits or near-misses is given on the vertical axis. FIG. 11 shows that the maraviroc oil-blended SDNs with soybean oil formed good nanodispersions when the combination of hydrophilic polymer and surfactant used was HPMC and TPGS.

FIG. 12 shows the Pₐₚₚ ratio of aqueous maraviroc (“Conventional MVC”), conventional maraviroc SDN (“Nanodispersion 1”) and maraviroc oil-blended SDN (“Nanodispersion 2”) as determined in Example 12. Monolayers were incubated for 1 h at 37° C., 5% CO₂. *, P<0.05 (Two-tailed unpaired t-test) (±SD, n=4).

FIG. 13 shows exposure curves for aqueous maraviroc (“Conventional MVC”) and a conventional maraviroc SDN (“Nanodispersion 1”) as outlined in Example 13. The conventional maraviroc SDN exhibits a 2.4- and 2.5-fold increase in AUC₀₋₄ and Cₐᵥₑ, respectively, and a 1.65-fold reduction in the Cₘₐₓ:Cₘᵢₙ ratio compared to the aqueous maraviroc preparation.

FIG. 14 shows exposure curves for aqueous maraviroc (“Conventional MVC”) and a maraviroc oil-blended SDN (“Nanodispersion 2”) as outlined in Example 13. The conventional maraviroc SDN exhibits a 2.4-, 2.8- and 4.5-fold increase in AUC₀₋₄, Cₐᵥₑ and Cₘₐₓ:Cₘᵢₙ ratio, respectively, for the maraviroc oil-blended SDN compared to the aqueous maraviroc preparation.

FIG. 15 shows a bar chart for the concentration of maraviroc in various tissues following the oral administration of various maraviroc containing compositions as per Example 13. *, P<0.05; **, P<0.01; ***, P<0.001 (Unpaired two-tailed t-test) (±SD, n=4).

FIG. 16 shows a bar chart displaying the relative maraviroc release rate constants as measured by RED over 6 hours for aqueous maraviroc and a number of oil-blended SDNs, as described in Example 14. RED plates incubated at 37° C., 100 rpm. (P=<0.001; unpaired two-tailed t-test).

FIG. 17 shows exposure curves for aqueous maraviroc (“Conventional”) and three maraviroc oil-blended SDNs (Nanodispersions 1, 2 and 3) as outlined in Example 15. The curves show significantly enhanced performance for Nanodispersions 1 and 3, and comparable performance Nanodispersion 2.

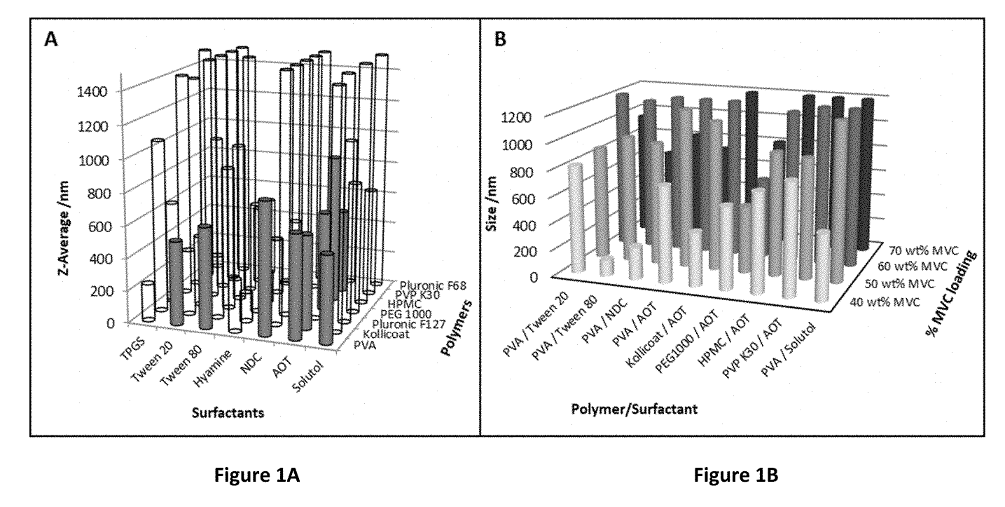

### DETAILED DESCRIPTION OF THE INVENTION

### Definitions

The term “nanoparticle” or “nanoparticulate” is used herein to mean a particle having a Z-average diameter of less than or equal to 1 micron (μm). The term Z-average diameter is taken to mean the Z-average diameter as determined by Dynamic Light Scattering (DLS).

The term “maraviroc” is used herein to refer to maraviroc, commonly used in HIV treatments, and includes pharmaceutically acceptable salts and solvates thereof, as well as any polymorphic or amorphous forms thereof.

It is to be appreciated that references to “preventing” or “prevention” elate to prophylactic treatment and includes preventing or delaying the appearance of clinical symptoms of the state, disorder or condition developing in a human that may be afflicted with or predisposed to the state, disorder or condition but does not yet experience or display clinical or subclinical symptoms of the state, disorder or condition.

It will be appreciated that references to “treatment” or “treating” of a state, disorder or condition includes: (1) inhibiting the state, disorder or condition, i.e., arresting, reducing or delaying the development of the disease or a relapse thereof (in case of maintenance treatment) or at least one clinical or subclinical symptom thereof, or (2) relieving or attenuating the disease, i.e. causing regression of the state, disorder or condition or at least one of its clinical or subclinical symptoms.

A “therapeutically effective amount” means the amount of a compound that, when administered to a mammal for treating a disease, is sufficient to effect such treatment for the disease. The “therapeutically effective amount” will vary depending on the compound, the disease and its severity and the age, weight, etc., of the patient to be treated.

It is to be understood that the term “oil” it is to be interpreted as a liquids of biological origin which are immiscible with water. It is to be further understood that the term also encompasses versions of such liquids which are produced synthetically or chemically modified (e.g. by hydrogenation).

The term “volatile oil” refers to the water-immiscible solvent which forms the oil phase of the water in oil emulsion and is then removed during the drying step, as well as to the oil phase of said emulsion itself.

To be clear, it should be understood that the “volatile oil” and the “oil” are distinct entities. For example, where the process for the formation of the solid compositions refers to forming an oil in water emulsion, the oil used is the volatile oil, not the oil. As a further example, when said emulsion is then dried, it is the volatile oil that is removed, not the oil.

The term “water-insoluble active” and like terms (e.g. “active”) are to be interpreted as referring to a compound with biological activity. This activity may be pharmacological or biocidal in nature.

### Solid Active Composition

The present invention provides a solid composition, comprising nanoparticles comprising active dispersed within a mixture of at least one hydrophilic polymer and at least one surfactant. Optionally the nanoparticles may consist essentially of active. Further optionally the nanoparticles may consist of active. Optionally the nanoparticles may consist essentially of active, hydrophilic polymer and/or surfactant. Further optionally the nanoparticles may consist of active, hydrophilic polymer and/or surfactant. Thus, in a further preferred embodiment, the invention also provides a solid maraviroc composition, comprising nanoparticles where each of said nanoparticles comprises maraviroc dispersed within a mixture of at least one hydrophilic polymer and at least one surfactant.

The nanoparticles comprising active substance are dispersed within a solid excipient mixture comprising the hydrophilic polymer and the surfactant.

The solid composition of the present invention may be administered as it is to a patient, or further formulated to provide a pharmaceutical composition in the form of, for example, a tablet, capsule, lozenge, or a dispersible powder or granule formulation.

The nanoparticles comprising active have a Z-average particle diameter of less than or equal to 1 micron (μm). In a particular embodiment, the nanoparticles comprising active have a Z-average particle diameter of between 100 and 1000 nm. In another embodiment, the nanoparticles comprising active have a Z-average particle diameter between 100 and 800 nm. In another embodiment, the nanoparticles comprising active have a Z-average particle diameter between 100 and 700 nm. In yet another embodiment, the nanoparticles comprising active have a Z-average particle diameter between 100 and 600 nm. The nanoparticles comprising active may comprise active, optionally the nanoparticles comprising active may consist essentially of active, further optionally the nanoparticles comprising active may consist of active.

The Z-average particle diameter of the nanoparticles may be assessed by any suitable technique known in the art (e.g. laser diffraction, laser scattering, electron microscopy). In an embodiment of the invention, a Z-average particle diameter is assessed by dispersing the solid composition in an aqueous medium and determining the particle diameter with a Zetasizer (Malvern Instruments Ltd), a DLS instrument.

In an embodiment, the polydispersity of the nanoparticles comprising active is less than or equal to 0.8, suitably less than or equal to 0.6, and most suitably less than or equal to 0.5. The polydispersity relates to the diameter of the active nanoparticles and may be determined by suitable techniques known in the art (e.g. laser diffraction, laser scattering, electron microscopy). In an embodiment of the present invention, the polydispersity of particle diameters of the nanoparticles comprising active may be suitably assessed with a Malvern Zetasizer (Malvern Instruments Ltd).

In a particular embodiment, the average zeta potential of the nanoparticles comprising active when dispersed in an aqueous medium is between −100 and +100 mV. In another embodiment, the zeta potential of the nanoparticles comprising active is between −25 and +25 mV. In another embodiment, the zeta potential of the nanoparticles comprising active is between −20 and +20 mV. In yet another embodiment, the zeta potential of the nanoparticles comprising active is between −25 and 0 mV. In general it has been found that zeta potentials of a relatively small magnitude (either positive or negative) allow the nanoparticles to better penetrate into and accumulate within cells. In accordance with the present invention, average zeta potentials can be measured by techniques known in the art, such as using a Zetasizer (Malvern Instruments Ltd).

The solid composition may comprise particles or granules of larger size, for example, 5 to 30 microns (μm) in size, but each particle or granule contains a plurality of nanoparticles comprising active dispersed within a mixture of the hydrophilic polymer and surfactant. Furthermore, these larger particles or granules disperse when the solid composition is mixed with an aqueous medium to form discrete nanoparticles comprising active.

In an embodiment, the solid composition comprises a single hydrophilic polymer and a single surfactant selected from those listed herein. In an alternative embodiment, the solid composition comprises two or more hydrophilic polymers and/or two or more surfactant selected from those listed herein may be present.

### Hydrophilic Polymer

A wide range of hydrophilic polymers are suitable for use in pharmaceutical formulations. Examples of such polymers include:

(a) homo or co-polymers of monomers selected from: vinyl alcohol, acrylic acid, methacrylic acid, acrylamide, methacrylamide, acrylamide aminoalkylacrylates, aminoalkyl-methacrylates, hydroxyethylacrylate, methylpropane sulphonates, hydroxyethylmethylacrylate, vinyl pyrrolidone, vinyl imidazole, vinyl amines, vinyl pyridine, ethyleneglycol, propylene glycol, ethylene oxides, propylene oxides, ethyleneimine, styrenesulphonates, ethyleneglycolacrylates and ethyleneglycol methacrylate;
(b) polyvinyl alcohol (PVA), a polyvinyl alcohol-polyethylene glycol graft copolymer, a block copolymer of polyoxyethylene and polyoxypropylene, polyethylene glycol, and polyvinylpyrrolidone, or a combination thereof;
(c) cellulose derivatives, for example, cellulose acetate, methylcellulose, methyl-ethylcellulose, hydroxy-ethylcellulose, hydroxy-ethylmethyl-cellulose, hydroxy-propylcellulose (HPC), hydroxy-propylmethylcellulose (HPMC), hydroxy-propylbutylcellulose, ethylhydroxy-ethylcellulose, carboxy-methylcellulose and its salts (eg the sodium salt—SCMC), or carboxy-methylhydroxyethylcellulose and its salts (for example the sodium salt);
(d) gums, such as, guar gum, alginate, locust bean gum and xanthan gum;
(e) polysaccharides, such as, dextran, xyloglucan and gelatin (or hydrolysed gelatin);
(f) cyclodextrins, such as, beta-cyclodextrin;
(g) mixtures thereof.

Copolymers may be statistical copolymers (also known as a random copolymer), a block copolymer, a graft copolymer or a hyperbranched copolymer. Additional co-monomers may also be present provided that their presence does not effect the water-solubility of the resulting polymeric material.

Particular examples of homopolymers include poly-vinylalcohol (PVA), poly-acrylic acid, poly-methacrylic acid, poly-acrylamides (such as poly-N-isopropylacrylamide), poly-methacrylamide; poly-acrylamines, poly-methyl-acrylamines, (such as polydimethylaminoethylmethacrylate and poly-N-morpholinoethylmethacrylate), polyvinylpyrrolidone (PVP), poly-styrenesulphonate, polyvinylimidazole, polyvinylpyridine, poly-2-ethyl-oxazoline poly-ethyleneimine and ethoxylated derivatives thereof.

In the present invention, the hydrophilic polymer is selected from those hydrophilic polymers that are capable of stabilising nanoparticles comprising active in an aqueous dispersion together with a surfactant as defined herein, and which are also suitable for pharmaceutical use (e.g. they are approved for use by the US Food and Drug Administration).

The hydrophilic polymer is a pharmaceutically acceptable hydrophilic polymer.

It shall be appreciated that any molecular weight (Mw) or molecular number (Mn) values quoted herein span the range of Mw and Mn values that may be present in the polymer.

In a particular embodiment, the polyvinyl alcohol has an average molecular weight between 5000 and 200000 Da, suitably with a 75-90% hydrolysis level (i.e. % free hydroxyls). In a particular embodiment, the polyvinyl alcohol has a 75-90% hydrolysis level. In another embodiment, the polyvinyl alcohol has a 75-85% hydrolysis level. In a particular embodiment, the polyvinyl alcohol has an average molecular weight between 9000 and 10000 Da, suitably with an 80% hydrolysis level. In a particular embodiment, the polyvinyl alcohol has a 75-90% hydrolysis level, suitably a 75-85% hydrolysis level.

In a particular embodiment, the polyvinyl alcohol-polyethylene glycol graft copolymer has an average molecular weight between 30000 and 60000 Da, suitably with a PVA/PEG ratio of between 90:10 and 25:75. In a particular embodiment, the polyvinyl alcohol-polyethylene glycol graft copolymer has an average molecular weight between 40000 and 50000 Da, suitably with a PVA/PEG ratio of between 85:15 and 25:75. Suitably the polyvinyl alcohol-polyethylene glycol graft copolymer has a PVA/PEG ratio of between 90:10 and 25:75, more suitably a PVA/PEG ratio of between 85:15 and 25:75. In a particular embodiment, the polyvinyl alcohol-polyethylene glycol graft copolymer is a Kollicoat® polymer (supplied by BASF, Kollicoat® is a polyvinyl alcohol-polyethylene glycol graft copolymer with a PVA/PEG ratio of 75:25). In a particular embodiment, the Kollicoat® is Kollicoat® Protect (supplied by BASF, Kollicoat® Protect is a mixture of PVA (35-45 wt %) and polyvinyl alcohol-polyethylene glycol graft copolymer with a PVA/PEG ratio of 75:25 (55-65 wt %)).

The block copolymer of polyoxyethylene and polyoxypropylene is suitably either a diblock copolymer of polyoxyethylene and polyoxypropylene or a triblock copolymer thereof. In a particular embodiment, the block copolymer of polyoxyethylene and polyoxypropylene is a Poloxamer.

A “poloxamer” is a non-ionic triblock copolymer comprising a central hydrophobic chain of polyoxypropylene, and hydrophilic chains of polyoxyethylene either side of this central hydrophobic chain. A “poloxamer” is typically named with the letter “P” followed by three numerical digits (e.g. P407), where the first two digits multiplied by 100 gives the approximate molecular mass of the polyoxypropylene chain, and the third digit multiplied by 10 provides the percentage polyoxyethylene content of the poloxamer. For example, P407 is a poloxamer having a polyoxypropylene molecular mass of about 4,000 g/mol and a polyoxyethylene content of about 70%. Poloxamers are also known as Pluronics®, as well as by several other commercial names.

The Poloxamer is suitably a pharmaceutically acceptable Poloxomer such as Poloxamer P407 or Poloxamer P188.

In a particular embodiment, the polyethylene glycol (PEG) has an average molecular weight of 500 to 20000 Da. In a particular embodiment, the polyethylene glycol is PEG 1K (i.e. with an average molecular weight of about 1000 Da).

In a particular embodiment, the HPMC has an average molecular weight of 10000 to 400000 Da. In a particular embodiment, the HPMC has an average molecular weight of about 10000.

In a particular embodiment, the polyvinylpyrrolidone has an average molecular weight of 2000 to 1,000,000 Da. In a particular embodiment, the polyvinylpyrrolidone has an average molecular weight of 30000 to 55000 Da. In a particular embodiment the polyvinylpyrrolidone is polyvinylpyrrolidone K30 (PVP K30).

### Hydrophilic Polymers for Solid Maraviroc Compositions

In embodiments of the present invention where the solid compositions is a solid maraviroc composition comprising nanoparticles including maraviroc dispersed within a mixture of at least one hydrophilic polymer and at least one surfactant, the hydrophilic polymer may be drawn from any of the aforementioned pharmaceutically acceptable hydrophilic polymers.

In a particular embodiment, the hydrophilic polymer is selected from polyvinyl alcohol (PVA), a polyvinyl alcohol-polyethylene glycol graft copolymer, polyethylene glycol, hydroxypropyl methyl cellulose (HPMC) and polyvinylpyrrolidone (PVP), or a combination thereof.

In a particular embodiment, the hydrophilic polymer is selected from PVA, a Kollicoat®, PEG 1K, HPMC and PVP K30, or a combination thereof.

In a particular embodiment, the hydrophobic polymer is selected from PVA, a Kollicoat® and HPMC, or a combination thereof.

In a particular embodiment, the hydrophobic polymer is selected from PVA, a Kollicoat® and HPMC.

In a particular embodiment, the hydrophilic polymer is PVA.

### Hydrophilic Polymers for Solid Compositions Additionally Comprising Oil

In embodiments of the present invention where the solid compositions comprise nanoparticles comprising at least one water-insoluble active and at least one oil dispersed within a mixture including at least one hydrophilic polymer and at least one surfactant, the hydrophilic polymer may be drawn from any of the aforementioned pharmaceutically acceptable hydrophilic polymers.

In embodiments, the hydrophilic polymer is selected from polyvinyl alcohol (PVA), polyvinyl alcohol-polyethylene glycol graft copolymer, polyethylene glycol, a block copolymer of polyoxyethylene and polyoxypropylene hydroxypropyl methyl cellulose (HPMC) and polyvinylpyrrolidone (PVP), or a combination thereof.

In a particular embodiment, the hydrophilic polymer is selected from polyvinyl alcohol (PVA), polyvinyl alcohol-polyethylene glycol graft copolymer, polyethylene glycol, a block copolymer of polyoxyethylene and polyoxypropylene hydroxypropyl methyl cellulose (HPMC) and polyvinylpyrrolidone (PVP).

In a particular embodiment, the hydrophilic polymer is selected from PVA, Kollicoat®, PEG 1K, or a combination thereof.

In a particular embodiment, the hydrophilic polymer is selected from PVA, Kollicoat®, PEG 1K, PVP K30, a block copolymer of polyoxyethylene and polyoxypropylene, or a combination thereof.

In a particular embodiment, the hydrophilic polymer is selected from PVA, HPMC or a combination thereof.

In a particular embodiment, the hydrophilic polymer is selected from PVA or HPMC. Such polymers may find particular use in formulations containing around 50 wt % maraviroc, especially if such formulations comprise soy bean oil or Vitamin E. Such polymers may also find particular use in formulations containing around 60 wt % maraviroc, especially if such formulations comprise soy bean oil.

In a particular embodiment, the hydrophilic polymer is HPMC. This polymer may find particular use in formulations containing around 70 wt % maraviroc, especially if such formulations comprise soy bean oil.

### Surfactant

Surfactants suitable for pharmaceutical use may be:
non-ionic (e.g. ethoxylated triglycerides; fatty alcohol ethoxylates; alkylphenol ethoxylates; fatty acid ethoxylates; fatty amide ethoxylates; fatty amine ethoxylates; sorbitan alkanoates; ethylated sorbitan alkanoates; alkyl ethoxylates; Pluronics™; alkyl polyglucosides; stearol ethoxylates; alkyl polyglycosides; sucrose fatty acid esters, anionic, cationic, amphoteric or zwitterionic);
anionic (e.g. alkylether sulfates; alkylether carboxylates; alkylbenzene sulfonates; alkylether phosphates; dialkyl sulfosuccinates (e.g. dioctyl sodium sulfosuccinate (AOT)); sarcosinates; alkyl sulfonates; soaps; alkyl sulfates; alkyl carboxylates (e.g. sodium deoxycholate (NDC); alkyl phosphates; paraffin sulfonates; secondary n-alkane sulfonates; alpha-olefin sulfonates; isethionate sulfonates; alginates);
cationic (e.g. fatty amine salts; fatty diamine salts; quaternary ammonium compounds; phosphonium surfactants; sulfonium surfactants; sulfonxonium surfactants); or
zwitterionic (e.g. N-alkyl derivatives of amino acids (such as glycine, betaine, aminopropionic acid); imidazoline surfactants; amine oxides; amidobetaines).

In the present invention, the surfactant is suitably selected from those surfactants that are capable of stabilising nanoparticles comprising active together with a hydrophilic polymer as defined herein, and which are also approved for pharmaceutical use (e.g. they are approved for use by the US Food and Drug Administration).

It will be appreciated that the hydrophilic polymer and the surfactant may both be PVA. In other words, the PVA may function as both the surfactant and the hydrophilic polymer. The total amount of PVA that may be present in such circumstances is that defined hereinafter for the total of the surfactant and hydrophilic polymer.

In a particular embodiment, the polyoxyethylene sorbitan fatty acid ester is selected from polysorbate 20 (commercially available as Tween® 20) and polysorbate 80 (commercially available as Tween® 80).

In a particular embodiment, the polyethylenglycol-12-hydroxystearate has a molecular weight of 300 to 3000 Da. In a particular embodiment, the polyethylenglycol-12-hydroxystearate has a molecular weight of 600 to 700 Da (e.g. commercially available as Solutol® HS).

### Surfactants for Solid Maraviroc Compositions

In embodiments of the present invention where the solid compositions is a solid maraviroc composition comprising nanoparticles including maraviroc dispersed within a mixture of at least one hydrophilic polymer and at least one surfactant, the surfactants may be drawn from any of the aforementioned pharmaceutically acceptable surfactants.

In a particular embodiment, the surfactant is selected from a polyoxyethylene sorbitan fatty acid ester, sodium deoxycholate, dioctyl sodium sulfosuccinate and polyethyleneglycol-12-hydroxystearate, polyvinyl alcohol (PVA) or a combination thereof

In a particular embodiment, the surfactant is selected from polysorbate 80, sodium deoxycholate, dioctyl sodium sulfosuccinate, or a combination thereof.

In a particular embodiment, the surfactant is selected from polysorbate 80, sodium deoxycholate, dioctyl sodium sulfosuccinate.

In a particular embodiment, the surfactant is dioctyl sodium sulfosuccinate.

### Surfactants for Solid Compositions Additionally Comprising Oil

In embodiments of the present invention where the solid compositions comprise nanoparticles comprising at least one water-insoluble active and at least one oil dispersed within a mixture of at least one hydrophilic polymer and at least one surfactant, the surfactants may be drawn from any of the aforementioned pharmaceutically acceptable surfactants.

In embodiments, the surfactant is selected from TPGS, a polyoxyethylene sorbitan fatty acid ester, sodium deoxycholate, dioctyl sodium sulfosuccinate and polyethyleneglycol-12-hydroxystearate, hyamine, polyvinyl alcohol (PVA) or a combination thereof.

In embodiments, the surfactant is selected from TPGS, a polyoxyethylene sorbitan fatty acid ester, sodium deoxycholate, dioctyl sodium sulfosuccinate and polyethyleneglycol-12-hydroxystearate, hyamine or polyvinyl alcohol (PVA).

In a particular embodiment, the surfactant is selected from TPGS, a polyoxyethylene sorbitan fatty acid ester, polyethyleneglycol-12-hydroxystearate or a combination thereof.

In a particular embodiment, the surfactant is selected from TPGS, a polyoxyethylene sorbitan fatty acid ester, sodium deoxycholate, or combinations thereof.

In a particular embodiment, the surfactant is selected from TPGS, a polyoxyethylene sorbitan fatty acid ester or sodium deoxycholate. Such surfactants may find particular use in formulations containing around 50 wt % maraviroc, especially if such formulations comprise Vitamin E or soy bean oil.

In a particular embodiment, the surfactant is selected from TPGS, sodium deoxycholate, or combinations thereof.

In a particular embodiment, the surfactant is selected from TPGS or sodium deoxycholate. Such surfactants may find particular use in formulations containing around 60 wt % maraviroc, especially if such formulations comprise soy bean oil.

In a particular embodiment, the hydrophilic polymer is HPMC. Such surfactants may find particular use in formulations containing around 70 wt % maraviroc, especially if such formulations comprise soy bean oil.

### Particular Combinations of Hydrophilic Polymer and Surfactant

### Particular Combinations of Hydrophilic Polymer and Surfactant for Solid Maraviroc Compositions

In embodiments, PVA is the hydrophilic polymer and the surfactant is selected from polysorbate 20, polysorbate 80, sodium deoxycholate, dioctyl sodium sulfosuccinate and polyethylenglycol-12-hydroxystearate; a polyvinyl alcohol-polyethylene glycol graft copolymer is the hydrophilic polymer and dioctyl sodium sulfosuccinate is the surfactant; polyethylene glycol is the hydrophilic polymer and dioctyl sodium sulfosuccinate; hydroxypropyl methyl cellulose is the hydrophilic polymer and dioctyl sodium sulfosuccinate is the surfactant; or polyvinylpyrrolidone is the hydrophilic polymer and dioctyl sodium sulfosuccinate is the surfactant.

In other embodiments, PVA is the hydrophilic polymer and the surfactant is selected from polysorbate 80, sodium deoxycholate, dioctyl sodium sulfosuccinate and polyethylenglycol-12-hydroxystearate; a polyvinyl alcohol-polyethylene glycol graft copolymer is the hydrophilic polymer and dioctyl sodium sulfosuccinate is the surfactant; or hydroxypropyl methyl cellulose is the hydrophilic polymer and dioctyl sodium sulfosuccinate is the surfactant.

In other embodiments, PVA is the hydrophilic polymer where the surfactant is selected from polysorbate 80, sodium deoxycholate and dioctyl sodium sulfosuccinate.

In a particular embodiment, the hydrophilic polymer is PVA and the surfactant is dioctyl sodium sulfosuccinate.

The above PVA combinations are particularly advantageous where the PVA has an average molecular weight between 9000 and 10000 Da, suitably with a 75-85% hydrolysis level.

### Particular Combinations of Hydrophilic Polymer and Surfactant for Solid Compositions Additionally Comprising Oil

In embodiments, the combination of polymer and surfactant is selected from the following list: PVA and TPGS; PVA and a polyoxyethylene sorbitan fatty acid ester; PVA and sodium deoxycholate; PVA and dioctyl sodium sulfosuccinate; PVA and polyethyleneglycol-12-hydroxystearate; PVA and hyamine; a polyvinyl alcohol-polyethylene glycol graft copolymer and TPGS; a polyvinyl alcohol-polyethylene glycol graft copolymer and a polyoxyethylene sorbitan fatty acid ester; a polyvinyl alcohol-polyethylene glycol graft copolymer and sodium deoxycholate; a polyvinyl alcohol-polyethylene glycol graft copolymer and dioctyl sodium sulfosuccinate; a polyvinyl alcohol-polyethylene glycol graft copolymer and polyethyleneglycol-12-hydroxystearate a polyvinyl alcohol-polyethylene glycol graft copolymer and hyamine; a polyvinyl alcohol-polyethylene glycol graft copolymer and polyvinyl alcohol (PVA); polyethylene glycol and TPGS; polyethylene glycol and a polyoxyethylene sorbitan fatty acid ester; polyethylene glycol and sodium deoxycholate; polyethylene glycol and dioctyl sodium sulfosuccinate; polyethylene glycol and polyethyleneglycol-12-hydroxystearate; polyethylene glycol and hyamine; polyethylene glycol and polyvinyl alcohol (PVA); a block copolymer of polyoxyethylene and polyoxypropylene and TPGS; a block copolymer of polyoxyethylene and polyoxypropylene and a polyoxyethylene sorbitan fatty acid ester; a block copolymer of polyoxyethylene and polyoxypropylene and sodium deoxycholate; a block copolymer of polyoxyethylene and polyoxypropylene and dioctyl sodium sulfosuccinate a block copolymer of polyoxyethylene and polyoxypropylene and polyethyleneglycol-12-hydroxystearate; a block copolymer of polyoxyethylene and polyoxypropylene and hyamine; a block copolymer of polyoxyethylene and polyoxypropylene and polyvinyl alcohol (PVA); hydroxypropyl methyl cellulose and TPGS; hydroxypropyl methyl cellulose and a polyoxyethylene sorbitan fatty acid ester; hydroxypropyl methyl cellulose and sodium deoxycholate hydroxypropyl methyl cellulose and dioctyl sodium sulfosuccinate hydroxypropyl methyl cellulose and polyethyleneglycol-12-hydroxystearate hydroxypropyl methyl cellulose and hyamine; hydroxypropyl methyl cellulose and polyvinyl alcohol (PVA); polyvinylpyrrolidone and TPGS; polyvinylpyrrolidone and a polyoxyethylene sorbitan fatty acid ester; polyvinylpyrrolidone and sodium deoxycholate; polyvinylpyrrolidone and dioctyl sodium sulfosuccinate; polyvinylpyrrolidone and polyethyleneglycol-12-hydroxystearate; polyvinylpyrrolidone and hyamine; polyvinylpyrrolidone and polyvinyl alcohol (PVA); hydroxypropyl cellulose and TPGS; hydroxypropyl cellulose and a polyoxyethylene sorbitan fatty acid ester; hydroxypropyl cellulose and sodium deoxycholate hydroxypropyl cellulose and dioctyl sodium sulfosuccinate hydroxypropyl cellulose and polyethyleneglycol-12-hydroxystearate hydroxypropyl cellulose and hyamine; hydroxypropyl cellulose and polyvinyl alcohol (PVA).

PVA and TPGS; hydroxypropyl methyl cellulose and TPGS; and hydroxypropyl methyl cellulose and a polyoxyethylene sorbitan fatty acid ester are combinations which may find particular use in formulations containing around 50 wt % maraviroc, especially if such formulations comprise vitamin E.

PVA and TPGS; hydroxypropyl methyl cellulose and TPGS; and PVA and sodium deoxycholate are combinations which may find particular use in formulations containing around 50 wt % maraviroc, especially if such formulations comprise soy bean oil.

PVA and TPGS and hydroxypropyl methyl cellulose and TPGS are combinations which may find particular use in formulations containing around 60 wt % maraviroc, especially if such formulations comprise soy bean oil.

Hydroxypropyl methyl cellulose and TPGS is a combination which may find particular use in formulations containing around 70 wt % maraviroc, especially if such formulations comprise soy bean oil.

### Oils for Solid Compositions Additionally Comprising Oil

The oil comprising the nanoparticles may be selected from natural oils, mineral oils, synthetic oils, silicone oils and mixtures thereof. Suitable oils may have a boiling point higher than that of the solvents.

Preferably the oil is a natural oil. Optionally, the natural oil is selected from peanut oil, soy bean oil, sesame oil, safflower oil, vegetable oil, avocado oil, rice bran oil, jojoba oil, Babassu oil, palm oil, coconut oil, castor oil, cotton seed oil, olive oil, flaxseed oil, rapeseed oil and mixtures thereof. Vitamin E may also considered to be a natural oil for the purposes of the present invention. Animal and plant waxes may also considered to be natural oils for the purposes of the present invention (e.g. beeswax, lanolin, carnauba wax, candellila wax, ouricury wax and the like)

Preferably the oil is biocompatible as this would enable the liquid composition to be used in biological settings, for example as use in a medicament.

The oil may have an effect on the pharmacokinetics of the solid composition, aqueous nanodispersion or a pharmaceutical composition containing either of the solid composition or aqueous nanodispersion.

The nanoparticles may contain one oil, preferably selected from those listed above. Alternatively, the nanoparticles may contain multiple oils, preferably selected separately from those listed above.

Preferred oils include soybean oil and Vitamin E.

Soybean oil is a preferred oil for compositions also comprising maraviroc. Vitamin E is a preferred oil for compositions also comprising maraviroc.

### Formulation of Solid Maraviroc Compositions

In a particular embodiment, the solid maraviroc composition as defined herein comprises 40 to 90 wt % of maraviroc, 40 to 80 wt % of maraviroc or 40 to 70 wt % of maraviroc. In another embodiment, the solid composition comprises 50 to 90 wt % of maraviroc, 50 to 80 wt % of maraviroc or 50 to 70 wt % of maraviroc. In another embodiment, the solid composition comprises 60 to 80 wt % of maraviroc, 65 to 75 wt % of maraviroc or around 70 wt % of maraviroc.

The solid maraviroc compositions of the present invention therefore permit high drug loadings, which keeps the potentially toxic excipients (e.g. surfactants) to a minimum.

Suitably, the solid maraviroc composition comprises 10 to 60 wt % of the hydrophilic polymer and surfactant combined, more suitably 20 to 60 wt %, even more suitably 25 to 50 wt %, most suitably 25 to 40 wt %. In a particular embodiment, the solid maraviroc composition comprises 25 to 35 wt % of the hydrophilic polymer and surfactant combined.

In a particular embodiment, the solid maraviroc composition comprises 5 to 50 wt % of hydrophilic polymer. In another embodiment, the solid maraviroc composition comprises 10 to 40 wt % of hydrophilic polymer. In another embodiment, the solid maraviroc composition comprises 15 to 30 wt % of hydrophilic polymer. In a particular embodiment, the solid maraviroc composition comprises 15 to 25 wt % of hydrophilic polymer.

In a particular embodiment, the solid maraviroc composition comprises 1 to 25 wt % of surfactant. In another embodiment, the solid maraviroc composition comprises 2 to 20 wt % of surfactant. In another embodiment, the solid maraviroc composition comprises 3 to 10 wt % of surfactant.

Where either PVA or the block copolymer of polyoxyethylene and polyoxypropylene serve both as the surfactant and the hydrophilic polymer, the abovementioned wt % values for the hydrophilic polymer and surfactant combined still apply. For example, where either PVA or the block copolymer of polyoxyethylene and polyoxypropylene serve both as the surfactant and the hydrophilic polymer, the solid composition suitably comprises 10 to 60 wt % of PVA or a block copolymer of polyoxyethylene and polyoxypropylene; more suitably 20 to 60 wt % of PVA or a block copolymer of polyoxyethylene and polyoxypropylene, even more suitably 25 to 50 wt % of PVA or a block copolymer of polyoxyethylene and polyoxypropylene, most suitably 25 to 40 wt % of PVA or a block copolymer of polyoxyethylene and polyoxypropylene. In a particular embodiment, the solid maraviroc composition comprises 25 to 35 wt % of PVA or a block copolymer of polyoxyethylene and polyoxypropylene.

In an embodiment, the solid maraviroc composition comprises the hydrophilic polymer and surfactant in a respective ratio in the range of 30:1 to 1:10. In a particular embodiment, the solid composition comprises the hydrophilic polymer and surfactant in a respective ratio in the range of 15:1 to 1:2. In another embodiment, the solid composition comprises the hydrophilic polymer and surfactant in a respective ratio in the range of 10:1 to 2:1. In a particular embodiment, the solid composition comprises the hydrophilic polymer and surfactant in a respective ratio in the range of 6:1 to 3:1.

In a particular embodiment, the solid maraviroc composition comprises:
40 to 80 wt % maraviroc;
10 to 40 wt % hydrophilic polymer; and
2 to 20 wt % surfactant.

In a particular embodiment, the solid maraviroc composition comprises 15-25 wt % PVA as the hydrophilic polymer, and 5-15 wt % dioctyl sodium sulfosuccinate (AOT) as the surfactant. In another embodiment, the solid maraviroc composition comprises 20-25 wt % PVA as the hydrophilic polymer, and 5-10 wt % dioctyl sodium sulfosuccinate as the surfactant. In a particularly preferred embodiment, the solid maraviroc composition comprises 18-22 wt % PVA as the hydrophilic polymer, and 8-12 wt % dioctyl sodium sulfosuccinate as the surfactant. Suitably, such compositions comprise 25 to 35 wt % of the hydrophilic polymer and surfactant combined, more suitably 28 to 32 wt %.

In a particular embodiment, the solid maraviroc composition comprises 15-25 wt % PVA as the hydrophilic polymer, and 5-15 wt % polysorbate 80 as the surfactant. In another embodiment, the solid maraviroc composition comprises 20-25 wt % PVA as the hydrophilic polymer, and 5-10 wt % polysorbate 80 as the surfactant. In a particularly preferred embodiment, the solid maraviroc composition comprises 18-22 wt % PVA as the hydrophilic polymer, and 8-12 wt % polysorbate 80 as the surfactant. Suitably, such compositions comprise 25 to 35 wt % of the hydrophilic polymer and surfactant combined, more suitably 28 to 32 wt %.

In a particular embodiment, the solid maraviroc composition comprises 15-25 wt % PVA as the hydrophilic polymer, and 5-15 wt % sodium deoxycholate as the surfactant. In another embodiment, the solid maraviroc composition comprises 20-25 wt % PVA as the hydrophilic polymer, and 5-10 wt % sodium deoxycholate as the surfactant. In a particularly preferred embodiment, the solid maraviroc composition comprises 18-22 wt % PVA as the hydrophilic polymer, and 8-12 wt % sodium deoxycholate as the surfactant. Suitably, such compositions comprise 25 to 35 wt % of the hydrophilic polymer and surfactant combined, more suitably 28 to 32 wt %.

Unless otherwise stated, the above weight percentages relate to the percentage (%) by weight of a particular constituent as a proportion of the total weight of the solid maraviroc composition.

The solid maraviroc composition may comprise one or more additional excipients, for instance, to further facilitate dispersion or stabilisation of dispersions of the nanoparticles either in a pharmaceutically acceptable diluent or in vivo.

### Formulation of Solid Compositions Additionally Comprising Oil

In a particular embodiment, the solid composition additionally comprising oil as defined herein comprises 40 to 90 wt % of active, 40 to 80 wt % of active or 40 to 70 wt % of active. In another embodiment, the solid composition additionally comprising oil comprises 50 to 90 wt % of active, 50 to 80 wt % of active or 50 to 70 wt % of active. In another embodiment, the solid composition additionally comprising oil comprises 60 to 80 wt % of active, 65 to 75 wt % of active or around 70 wt % of active.

In a particular embodiment, the solid composition additionally comprising oil as defined herein comprises 2 to 30 wt % oil, 4 to 20 wt % oil, 6 to 15 wt % oil or 8 to 12 wt % oil.

In a particular embodiment, the ratio of active to oil is in the range of 10:1 to 2:1, 8:1 to 4:1 or 6:1.

The solid compositions additionally comprising oil of the present invention therefore permit high drug loadings, which keeps the potentially toxic excipients (e.g. surfactants) to a minimum.

Suitably, the solid composition additionally comprising oil comprises 10 to 60 wt % of the hydrophilic polymer and surfactant combined, more suitably 20 to 60 wt %, even more suitably 25 to 50 wt %, most suitably 25 to 40 wt %. In a particular embodiment, the solid composition additionally comprising oil comprises 25 to 35 wt % of the hydrophilic polymer and surfactant combined.

In a particular embodiment, the solid composition additionally comprising oil comprises 5 to 50 wt % of hydrophilic polymer. In another embodiment, the solid composition additionally comprising oil comprises 10 to 40 wt % of hydrophilic polymer. In another embodiment, the solid composition additionally comprising oil comprises 15 to 30 wt % of hydrophilic polymer. In a particular embodiment, the solid composition additionally comprising oil comprises 15 to 25 wt % of hydrophilic polymer.

In a particular embodiment, the solid composition additionally comprising oil comprises 1 to 25 wt % of surfactant. In another embodiment, the solid composition additionally comprising oil comprises 2 to 20 wt % of surfactant. In another embodiment, the solid composition additionally comprising oil comprises 3 to 10 wt % of surfactant.

In a particular embodiment, the solid composition additionally comprising oil comprises 40-80 wt % active, 5-20 wt % oil, 5-40 wt % hydrophilic polymer and 5-20 wt % surfactant In another embodiment, the solid composition additionally comprising oil comprises 45-75 wt % active, 5-15 wt % oil, 5-35 wt % hydrophilic polymer and 5-15 wt % surfactant. In another embodiment, the solid composition additionally comprising oil comprises 50-70 wt % active, 8.33-11.67 wt % oil, 8.33-31.67 wt % hydrophilic polymer and 10 wt % surfactant.

In a particular embodiment, the solid composition additionally comprising oil comprises 50 wt % active, 8.33 wt % oil, 31.67 wt % hydrophilic polymer and 10 wt % surfactant.

In a particular embodiment, the solid composition additionally comprising oil comprises 60 wt % active, 10 wt % oil, 20 wt % hydrophilic polymer and 10 wt % surfactant.

In a particular embodiment, the solid composition additionally comprising oil comprises 70 wt % active, 11.67 wt % oil, 8.33 wt % hydrophilic polymer and 10 wt % surfactant.

Unless otherwise stated, the above weight percentages relate to the percentage (%) by weight of a particular constituent as a proportion of the total weight of the solid composition.

The solid composition may comprise one or more additional excipients, for instance, to further facilitate dispersion or stabilisation of dispersions of the nanoparticles either in a pharmaceutically acceptable diluent or in vivo.

### Processes for Preparing the Solid Composition

Solid compositions of the present invention may be prepared by a number of methods well known in the art.

For example, the solid composition may be prepared by milling a solid form of the active. The milling may occur in the presence of the hydrophilic polymer and surfactant, or, alternatively, they may be mixed with the milled drug after the milling step.

However, it is generally preferred that the solid active compositions of the present invention are prepared by an oil in water emulsion technique using a volatile oil whereby the active is dissolved in the oil phase and the hydrophilic polymer and surfactant are present in the water phase. The volatile oil and water solvents are then removed by freeze drying, spray drying or spray granulation to provide a solid composition according to the invention.

Thus, in accordance with the present invention, there is provided a process for preparing a solid composition as defined herein, the process comprising:

(a) preparing an oil in water emulsion using a volatile oil comprising:
an oil phase comprising active; and
an aqueous phase comprising a hydrophilic polymer and a surfactant, each as defined herein; and

(b) removing the volatile oil and water to form the solid composition.

An advantage of the process of the present invention is that the emulsions formed in step (a) are sufficiently homogenous and stable to allow for effective and uniform drying in step (b). Furthermore, the nanoparticles formed are substantially uniform in their physical form (size, shape etc.).

Step (a) may be performed by methods well-known in the art. Any suitable method for forming the oil in water emulsion using a volatile oil defined in step (a) may therefore be used. In particular, the mixing of the volatile oil and water phases to form the volatile oil in water emulsion may be performed by methods well known in the art. For example, the mixing may involve stirring, sonication, homogenisation, or a combination thereof. In a particular embodiment, the mixing is facilitated by sonication and/or homogenisation.

Step (a) may be performed, for example, by using the methods described in WO 2004/011537 A1 (COOPER et al), which is hereby duly incorporated by reference.

In a particular embodiment, step (a) comprises:
(i) providing a volatile oil phase comprising active;
(ii) providing an aqueous phase comprising the hydrophilic polymer and surfactant; and
(iii) mixing the oil phase and aqueous phase to produce the oil in water emulsion.

Suitably, the volatile oil phase is provided by dissolving active in a suitable organic solvent.

Suitably, the aqueous phase is provided by dissolving hydrophilic polymer and surfactant in an aqueous medium, preferably in water. Alternatively the aqueous phase may be provided by mixing two separately prepared aqueous solutions of the surfactant and hydrophilic polymer.

In a particular embodiment, further aqueous medium (e.g. water) or organic solvent is added before or during mixing step (iii).

The concentration of active in the oil in water emulsion is suitably as concentrated as possible to facilitate effective scale-up of the process. For example, the concentration of active in the oil phase is suitably 5 to 75 mg/ml, more suitably 10 to 70 mg/ml.

The concentration of the hydrophilic polymer in the aqueous/water phase is suitably 0.5-50 mg/mL, more suitably 10 to 30 mg/ml.

The concentration of the surfactant in the aqueous/water phase emulsion is suitably 0.5 to 50 mg/mL, more suitably 10 to 30 mg/ml.

The organic solvent forming the oil phase is (substantially) immiscible with water. Suitably the organic solvent is aprotic. Suitably the organic solvent has a boiling point less than 120° C., suitably less than 100° C., suitably less than 90° C.

In a particular embodiment, the organic solvent is a selected from the Class 2 or 3 solvents listed in the International Conference on Harmonization (ICH) guidelines relating to residual solvents.

In a particular embodiment, the organic solvent is selected from chloroform, dichloromethane, dichloroethane, tetrachloroethane, cyclohexane, hexane(s), isooctane, dodecane, decane, methylbutyl ketone (MBK), methylcyclohexane, tetrahydrofuran, toluene, xylene, butyl acetate, mineral oil, tert-butylmethyl ether, heptanes(s), isobutyl acetate, isopropyl acetate, methyl acetate, methylethyl ketone, ethyl acetate, ethyl ether, pentane, and propyl acetate, or any suitably combination thereof.

In a particular embodiment, the organic solvent is selected from chloroform, dichloromethane, methylethylketone (MEK), methylbutylketone (MBK), and ethyl acetate.

In a particular embodiment the organic solvent is dichloromethane.

The volume ratio of aqueous phase to oil phase in mixing step (iii) is suitably between 20:1 and 1:4, more suitably between 10:1 and 1:1, most suitably between 6:1 and 2:1.

Mixing step (iii) suitably produces a substantially uniform oil in water emulsion. As previously indicated, mixing may be performed using methods well known in the art. Suitably, mixing step (iii) involves stirring, sonication, homogenisation, or a combination thereof. In a particular embodiment, mixing step (iii) involves sonication and/or homogenisation.

Step (b) may be performed using methods well known in the art. Suitably step (b) involves freeze drying, spray drying or spray granulation.

Step (b) may be performed using methods described in WO 2004/011537 A1 (COOPER et al), the entire contents of which are hereby incorporated by reference.

In a particular embodiment, step (b) involves freeze drying the oil in water emulsion. As such, step (b) may suitably comprise freezing the oil in water emulsion and then removing the solvents (i.e. the volatile oil and water) under vacuum.

Preferably, the freezing of the oil in water emulsion may be performed by externally cooling the oil in water emulsion. For example, a vessel containing the oil in water emulsion may be externally cooled, for example, by submerging the vessel in a cooling medium, such as liquid nitrogen. Alternatively the vessel containing the oil in water emulsion may be provided with an external “jacket” through which coolant is circulated to freeze the oil in water emulsion. Alternatively the vessel may comprise an internal element through which coolant is circulated in order to freeze the oil in water emulsion.

In a further alternative, the oil in water emulsion is frozen by being contacted directly with a cooling medium at a temperature effective for freezing the emulsion. In such cases, the cooling medium (e.g. liquid nitrogen) may be added to the oil in water emulsion, or the oil in water emulsion may be added to the cooling medium.

In a particular embodiment, the oil in water emulsion is added to the fluid medium (e.g. liquid nitrogen), suitably in a dropwise manner. This order of addition provides higher purities of final product. As such, frozen droplets of the oil in water emulsion may suitably form. Such frozen droplets may suitably be isolated (e.g. under vacuum to remove the fluid medium/liquid nitrogen). The solvent is then suitably removed from the frozen droplets under vacuum. The resulting solid composition is then isolated.

In an alternative aspect, the present invention provides a process for preparing a solid composition as defined herein, the process comprising:
(a) preparing a single phase solution comprising active, a hydrophilic polymer as defined herein, and a surfactant as defined herein, in one or more solvents; and
(b) spray-drying the mixture to remove the one or more solvents to form the solid composition.

In this aspect of the invention, the single phase solution comprising the active, hydrophilic polymer, and surfactant are all dissolved in one solvent or two or more miscible solvents. Such processes are described in WO 2008/006712, the entire contents of which are duly incorporated herein by reference. WO 2008/006712 also lists suitable solvents and combinations thereof for forming the single phase solution. In an embodiment, the single phase solution comprises two or more solvents (e.g. ethanol and water) which together solubilise the active, hydrophilic polymer, and the surfactant. In another embodiment, the single phase comprises a single solvent, for example ethanol or water. Removing of the one or more solvents from the single phase fluid mixture may involve spray drying—WO 2008/006712 details suitable spray-drying conditions.

In embodiments, the solvent(s) for the single phase solution is selected from lower (C1-C10) alcohols, such as methanol, ethanol, propanol, isopropanol, butanol, isobutanol, tertiary butanol, 1-pentanol; organic acids, such as formic acid, acetic acid; amides, such as formamide, N,N-dimethylformamide; nitriles, such as acetonitrile; or combinations thereof.

The present invention also provides a solid composition obtainable by, obtained by, or directly obtained by any of the processes described herein.

### Processes for Preparing Solid Maraviroc Compositions

In embodiments, processes for preparing solid maraviroc compositions are those described above, wherein the active is maraviroc and the hydrophilic polymer and surfactant are as described herein.

### Processes for Preparing Solid Compositions Additionally Comprising Oil

In embodiments, the process for preparing a solid composition comprising an oil includes an oil in water emulsion using a volatile oil as described above. In such embodiments, the oil is present in the non-aqueous phase of the emulsion.

In embodiments, the process for preparing a solid composition comprising an oil includes a single phase solution as described above. In such embodiments, the oil is present in the single phase solution.

Aqueous Dispersion

The present invention provides an aqueous dispersion, comprising a plurality of nanoparticles dispersed in an aqueous medium, the nanoparticles comprising the active, at least one hydrophilic polymer and at least one surfactant.

The present invention also provides an aqueous dispersion, obtainable by, obtained by, or directly obtained by dispersing the solid composition as defined herein in an aqueous medium. Suitably, an aqueous dispersion is prepared immediately prior to use.

When the solid composition is dispersed in the aqueous medium, the hydrophilic polymer and/or surfactant is dissolved within the aqueous medium to release the nanoparticles comprising maraviroc in a dispersed form. The nanoparticles comprising maraviroc, which were formerly dispersed within a solid mixture of the hydrophilic polymer and surfactant, then become dispersed within the aqueous medium in nanoparticulate form, whereby each nanoparticle includes maraviroc, the at least one hydrophilic polymer and the at least one surfactant. Without wishing to be bound by any particular theorem, a convenient way to visualise the maraviroc-containing nanoparticles may be to consider them as having an inner portion or core, and an outer section or coating. In this model, one may consider the core as comprising maraviroc, possibly also some hydrophilic polymer and/or surfactant, and the coating as comprising the hydrophilic polymer and/or surfactant, possibly including some maraviroc. The coating may be a continuous coating over a portion or the entirety of the surface of core. Alternatively, the coating may be a discontinuous coating over a portion or the entirety of the surface of the core. The association of the hydrophilic polymer(s) and surfactant(s) with the maraviroc in the nanoparticles may impart stability to the nanoparticles, thereby preventing premature coagulation and aggregation.

Suitably the relative amounts (including ratios) of maraviroc, hydrophilic polymer(s), and surfactant(s) are the same as defined above in relation to the solid composition. However, the skilled person will readily appreciate that their respective wt % values in the aqueous dispersion as a whole must be adjusted to take account of the aqueous medium. In a particular embodiment, the aqueous medium comprises 20 to 99.5 wt % of the total aqueous dispersion. In a particular embodiment, the aqueous medium comprises 50 to 98 wt % of the total aqueous dispersion. In a particular embodiment, the aqueous medium comprises 70 to 95 wt % of the total aqueous dispersion. Suitably, the remaining proportion of the aqueous dispersion comprises or essentially consists of the components of the solid maraviroc composition as defined above in relation to the solid composition forming the first aspect of the present invention, whose proportions within the aqueous dispersion as a whole are accordingly calculated (and scaled) by reference to the proportions recited in relation to the solid composition. For example, the remaining proportion of the aqueous dispersion may comprise or consist essentially of maraviroc, one or more hydrophilic polymer, one or more surfactant and optionally one or more additional anti-retroviral and/or anti-microbial agent, whose proportions within the aqueous dispersion as a whole are accordingly calculated (and scaled) by reference to the proportions recited in relation to the solid composition.

In a particular embodiment, the aqueous medium is water. In an alternative embodiment, the aqueous medium comprises water and one or more additional pharmaceutically acceptable diluents or excipients.

Aqueous dispersions of the present invention are advantageously stable for prolonged periods, both in terms of chemical stability and the stability of the particles themselves (i.e. with respect to aggregation, coagulation, etc.).

Aqueous dispersions of the present invention may be considered as pharmaceutical compositions of the present invention.

Aqueous dispersions of the present invention allow a measured aliquot to be taken therefrom for accurate dosing in a personalised medicine regime.

The particle diameter, polydispersity and zeta potential of the nanoparticles comprising maraviroc in the aqueous dispersion is as defined hereinbefore in relation to the solid composition. It will of course be appreciated that the particle diameter, polydispersity and zeta potential nanoparticles comprising maraviroc present in the solid composition are measured by dispersing the solid composition in an aqueous medium to thereby form an aqueous dispersion of the present invention.

In an embodiment, the aqueous dispersion comprises a single hydrophilic polymer and a single surfactant selected from those listed herein. In an alternative embodiment, the aqueous dispersion comprises two or more hydrophilic polymers and/or two or more surfactant selected from those listed herein.

### Aqueous Nanodispersions of Nanoparticles Including Maraviroc

In embodiments where the active is maraviroc the aqueous dispersion comprises a plurality of nanoparticles dispersed in an aqueous medium, the nanoparticles comprising maraviroc, at least one hydrophilic polymer and at least one surfactant

wherein the hydrophilic polymer is selected from polyvinyl alcohol, a polyvinyl alcohol-polyethylene glycol graft copolymer, polyethylene glycol, hydroxypropyl methyl cellulose and polyvinylpyrrolidone, or a combination thereof; and

wherein the surfactant is selected from a polyoxyethylene sorbitan fatty acid ester, sodium deoxycholate, dioctyl sodium sulfosuccinate, and polyethyleneglycol-12-hydroxystearate, polyvinyl alcohol (PVA), or a combination thereof.

### Aqueous Nanodispersions of Nanoparticles Including Oil

In embodiments where the nanoparticles additionally comprise an oil, the aqueous dispersion comprising a plurality of nanoparticles dispersed in an aqueous medium, the nanoparticles comprising the active, the oil at least one hydrophilic polymer and at least one surfactant.

In embodiments, the aqueous nanodispersion may comprise nanoparticles containing more than one oil, more than one active, more than one surfactant and/or more than one hydrophilic polymer.

In embodiments, the aqueous nanodispersion may comprise nanoparticles which contain different oils, active, surfactants and/or nanoparticles.

### Process for Preparing an Aqueous Dispersion

The aqueous dispersion may be formed by methods well known in the art. For example, active may be milled in the presence of an aqueous mixture of the hydrophilic polymer and surfactant.

In a particular aspect of the invention, however, there is provided a process for preparing an aqueous dispersion, comprising dispersing a solid active composition as defined herein in an aqueous medium. In embodiments the active is maraviroc. In embodiments the solid composition additionally comprises an oil as described herein.

In a particular embodiment, the aqueous medium is water. In an alternative embodiment, the aqueous medium comprises water and one or more additional excipients.

Dispersing the solid composition in the aqueous medium may comprise adding the solid composition to an aqueous medium and suitably agitating the resulting mixture (e.g. by shaking, homogenisation, sonication, stirring, etc.).

### Pharmaceutical Compositions

The present invention provides a pharmaceutical composition comprising a solid composition or an aqueous dispersion as defined herein. The pharmaceutical compositions of the present invention may further comprise one or more additional pharmaceutically acceptable excipients.

In embodiments the active is maraviroc. In embodiments the solid composition additionally comprises an oil as described herein.

The solid compositions of the invention may be formulated into a form suitable for oral use (for example as tablets, lozenges, hard or soft capsules, or dispersible powders or granules) by techniques known in the art. As such, the solid compositions of the invention may be mixed with one or more additional pharmaceutical excipients during this process, such as antiadherants, binders, coatings, enterics, disintegrants, fillers, diluents, flavours, colours, lubricants, glidants, preservatives, sorbents, and sweeteners.

In a particular embodiment, the pharmaceutical composition is a tablet or capsule comprising the solid composition.

The aqueous dispersion of the present invention may be administered as it is or further formulated with one or more additional excipients to provide a dispersion, elixir or syrup that is suitable for oral use, or a dispersion that is suitable for parenteral administration (for example, a sterile aqueous dispersion for intravenous, subcutaneous, intramuscular, intraperitoneal or intramuscular dosing).

In a particular embodiment, the pharmaceutical composition is an aqueous dispersion as described herein. Such dispersed formulations can be used to accurately measure smaller dosages, such as those suitable for administration to children.

In a particular embodiment, the pharmaceutical composition is in a form suitable for parenteral delivery, whether via intravenous or intramuscular delivery.

It will be appreciated that different pharmaceutical compositions of the invention may be obtained by conventional procedures, using conventional pharmaceutical excipients, well known in the art.

The pharmaceutical compositions of the invention contain a therapeutically effective amount of active. A person skilled in the art will know how to determine and select an appropriate therapeutically effective amount of active to include in the pharmaceutical compositions of the invention.

### Uses of the Nanoparticles Formulation and Pharmaceutical Composition

The present invention provides a solid composition or an aqueous dispersion as defined herein for use as a medicament.

In a particular aspect, the present invention further provides a solid composition or an aqueous dispersion as defined herein for use in the treatment and/or prevention of retroviral infections (e.g. HIV).

The present invention further provides a use of a solid composition or an aqueous dispersion as defined herein in the manufacture of a medicament for use in the treatment and/or prevention of retroviral infections (e.g. HIV).

The present invention further provides a method of treating and/or preventing a retroviral infection (e.g. HIV), the method comprising administering a therapeutically effective amount of a solid composition, an aqueous dispersion, or a pharmaceutical composition as defined herein, to a patient suffering from or at risk of suffering from a retroviral infection.

The term “retrovirus” generally refers to an RNA virus capable of self-duplication in a host cell using the reverse transcriptase enzyme to transcribe its RNA genome into DNA. The DNA is then potentially incorporated into the host's genome so that the virus can then replicate thereafter as part of the host's DNA.

The retroviral infection to be treated or prevented is suitably selected from human immunodeficiency virus (HIV), Alpharetrovirus, Betaretrovirus, Gammaretrovirus, Deltaretrovirus, Epsilonretrovirus, Lentivirus, Spumavirus, Metavirus, Errantvirus, Pseudovirus, Hepadnavirus, and Caulimovirus.

In a particular embodiment of the present invention, the retroviral infection to be treated or prevented is the human immunodeficiency virus (HIV), most suitably the human immunodeficiency virus (HIV) type 1.

The solid compositions, aqueous dispersions, and pharmaceutical compositions of the present invention are also suitably used to reduce the risk of or prevent HIV infection developing in subjects exposed to a risk of developing HIV infection.

Maraviroc, the active agent in the solid compositions, aqueous dispersion, and pharmaceutical compositions of the present invention, is an antiretroviral drug which acts as an entry inhibitor. As such, the solid composition, aqueous dispersion, and pharmaceutical compositions of the present invention are capable of inhibiting HIV from gaining entry to macrophages and T-cells. Moreover, the nanoparticles and pharmaceutical compositions of the present invention are suitable for use in antiretroviral therapies and prophylactic treatments.

Thus in another aspect of the invention there is provided a method of inhibiting HIV from gaining entry to a macrophage or a T-cell (in vivo or in vitro), the method comprising administering to said macrophage or a T-cell a solid composition, aqueous dispersion, or pharmaceutical composition as described herein.

In another aspect, the present invention provides a method of inhibiting HIV from gaining entry to a macrophage or a T-cell in a human or animal subject in need of such inhibition, the method comprising administering to said subject an effective amount of a solid composition, aqueous dispersion, or pharmaceutical composition as defined herein.

In another aspect, the present invention provides a solid composition, aqueous dispersion, or pharmaceutical composition as defined herein for use in the treatment of a disease or condition associated with HIV gaining entry to a macrophage or a T-cell.

In another aspect, the present invention provides the use of a solid composition, aqueous dispersion, or pharmaceutical composition as defined herein in the manufacture of a medicament for use in the treatment of a disease or condition associated with HIV gaining entry to a macrophage or a T-cell.

Maraviroc, the active agent in the solid compositions, aqueous dispersion, and pharmaceutical compositions of the present invention, also exhibits good lymphatic penetration compared to many antiretroviral agents.

Thus in another aspect of the invention, there is provided a method of inhibiting HIV from gaining entry to a macrophage or a T-cell within the lymphatic system of a human or animal subject in need of such inhibition, the method comprising administering to said subject an effective amount of a solid composition, aqueous dispersion, or pharmaceutical composition as defined herein.

In another aspect, the present invention provides a solid composition, aqueous dispersion, or pharmaceutical composition as defined herein for use in the treatment of a disease or condition associated with HIV gaining entry to a macrophage or a T-cell within the lymphatic system.

In another aspect, the present invention provides the use of a solid composition, aqueous dispersion, or pharmaceutical composition as defined herein in the manufacture of a medicament for use in the treatment of a disease or condition associated with HIV gaining entry to a macrophage or a T-cell within the lymphatic system.

### Routes of Administration

The solid compositions, aqueous dispersions, and pharmaceutical compositions of the invention may be administered to a subject by any convenient route of administration.

Routes of administration include, but are not limited to, oral (e.g. by ingestion); buccal; sublingual; transdermal (including, e.g., by a patch, plaster, etc.); transmucosal (including, e.g., by a patch, plaster, etc.); intranasal (e.g., by nasal spray); ocular (e.g., by eyedrops); pulmonary (e.g., by inhalation or insufflation therapy using, e.g., via an aerosol, e.g., through the mouth or nose); rectal (e.g., by suppository or enema); vaginal (e.g., by pessary); parenteral, for example, by injection, including subcutaneous, intradermal, intramuscular, intravenous, infraarterlal, intracardiac, intrathecal, intraspinal, intracapsular, subcapsular, intraorbital, intraperitoneal, intratracheal, subcuticular, intraarticular, subarachnoid, and intrasternal; or by implant of a depot or reservoir, for example, subcutaneously or intramuscularly.

In a particular embodiment (e.g. in HIV treatments), the route of administration is either oral or by implant of a depot or reservoir formulation.

### Combination Therapy

Although it is possible that the solid compositions, aqueous dispersions, and pharmaceutical compositions of the invention may be used as a sole medicament in the treatment and/or prevention of a retrovirus infection such as HIV, it is more typical that this agent will be used in combination with one or more additional anti-retroviral and/or anti-microbial agents. The combination of antiretroviral agents from different classes (i.e. with different mechanisms of action) is useful as such combinations are of greater efficacy and help to lower the incidence of drug-resistance.

Other antiretroviral agents suitable in combination treatments with the formulations and compositions of the present invention include Zidovudine, Zalcitabine, Didanosine, Stavudine, Lamivudine, Abacavir, Combivir (zidovudine+lamivudine), Trizivir (zidovudine+lamivudine+abacavir), Tenofovir, Emtricitabine, Truvada (Tenofovir+Emtricitabine), Epzicom/Kivexa (abacavir+lamivudine), Hydroxyurea, Nevirapine, Delavirdine, Etravirine, Rilpivirine, Atripla (lopinavir+emtricitabine+tenofovir), Indinavir, Ritonavir, Saquinavir, Nelfinavir, Amprenavir, Kaletra (lopinavir+ritonavir), Atazanavir, Fosamprenavir, Tipranavir, Darunavir, Enfuvirtide, Lopinavir, Raltegravir, Nevirapine, Efavirenz, Delavirdine, Etravirine, Rilprivrine, Artipla, Bictegravir, Cabotegravir, Dolutegravir, Elvitegravir, Raltegravir, 4′-ethynyl-2-fluoro-2′-deoxyadenosine (EFdA) or combinations thereof.

In embodiments the solid compositions, aqueous dispersions or pharmacological compositions of maraviroc according to the present invention are used in combination with antiretroviral agents which exhibit good lymphatic penetration. Said antiretroviral agents preferably use a different mechanism of action to maraviroc. The lymphatic system is a sanctuary site for HIV as many otherwise potent drugs have poor penetration into the lymphatic system. In other words, in infected patients the lymphatic system forms a reservoir for HIV, preventing the infection from being cleared. Maraviroc has good penetration into the lymphatic system, however, for effective treatment it is desirable to expose HIV to multiple drugs simultaneously. Therefore combining maraviroc compositions according to the present invention with further antiretroviral agents with good lymphatic penetration characteristics is advantageous as it would allow effective treatment of HIV in the lymphatic system.

In embodiments, maraviroc may be co-administered with up to 4 other agents in combination. Preferred combinations of agent to use in conjunction with maraviroc solid drug nanoparticles (SDNs) include:
i. Tenofovir disoproxil fumarate and Lamivudine;
ii. Tenofovir disoproxil fumarate and Emtricitabine;
iii. Tenofovir alafenamide and Lamivudine;
iv. Tenofovir alafenamide and Emtricitabine; and
v. Abacavir and Lamivudine.

Accordingly, an aspect of the invention provides a combination suitable for use in the treatment or prevention of a retrovirus infection, such as HIV, comprising a solid composition, an aqueous dispersion, or a pharmaceutical composition as defined hereinbefore, and one or more other antiretroviral agents.

The present invention also provides a solid composition, an aqueous dispersion, or a pharmaceutical composition as defined hereinbefore for use in the treatment or prevention of a retrovirus infection, such as HIV, wherein the solid composition, aqueous dispersion, or pharmaceutical composition is administered in combination with one or more other antiretroviral agents.

In a further aspect, the present invention provides a pharmaceutical composition comprising a solid maraviroc composition as defined herein and one or more additional antiretroviral agents.

In a further aspect, the present invention provides a pharmaceutical composition comprising an aqueous dispersion as defined herein which further comprises one or more additional antiretroviral agents.

Herein, where the term “combination” is used it is to be understood that this refers to simultaneous, separate or sequential administration. In one aspect of the invention “combination” refers to simultaneous administration. In another aspect of the invention “combination” refers to separate administration. In a further aspect of the invention “combination” refers to sequential administration. Where the administration is sequential or separate, the delay in administering the second component should not be such as to lose the beneficial effect of the combination.

Such conjoint treatment may be achieved by way of the simultaneous, sequential or separate dosing of the individual components of the treatment. Such combination products employ the formulations or compositions of this invention within the dosage range described hereinbefore and the other pharmaceutically-active agent within its approved dosage range.

In a further aspect of the invention, there is provided a pharmaceutical composition comprising a solid composition or an aqueous dispersion as defined herein; and one or more other antiretroviral agents. In a particular embodiment, the pharmaceutical composition is a single dosage form.

### Kit of Parts

The present invention provides a kit of parts comprising a solid composition as defined herein or pharmaceutical composition comprising the solid composition as defined herein, and a pharmaceutically acceptable aqueous diluent.

The solid composition or pharmaceutical composition comprising the solid composition as defined herein can be dispersed into the diluent to provide an aqueous dispersion as defined herein. Either the entire dispersion can then be administered, or a proportion of it can be measured and then administered (thereby providing a means of administering different dosages to individual patients).

In embodiments the active is maraviroc. In embodiments the solid composition additionally comprises an oil as described herein.

### EXAMPLES

The following examples describe the preparation of embodiments of maraviroc formulations according to the present invention, along with various analytical data.

### Example 1—Screening for Suitable Excipient Combinations

Aqueous stock solutions of polymer and surfactants were each prepared at concentrations of 22.5 mg ml⁻¹, a maraviroc solution was prepared was at a concentration of 70 mg ml⁻¹ in dichloromethane. Emulsions were prepared at a 4:1 water:dichloromethane ratio to form compositions comprising 30 wt % maraviroc, 55 wt % polymer and 15 wt % surfactant.

The initial screening consisted of a matrix of 49 samples which were prepared as above and lyophilised using a Virtis benchtop K freeze dryer for 48 hours to leave a dry porous product. Samples were immediately sealed until analysis.

The polymers and surfactants employed in this screen are detailed in Table 1A and Table 1B below:

TABLE 1A
List of 7 hydrophilic polymers initially screened

| | | m/dm\hat{ }3 |
|---|---|---|
| Polymer | MW | (22.5 mg/ml) |
| PEG 1000 | 1000 | 0.00225 |
| Pluronic F68 | 8400 | 0.000267857 |
| Pluronic F127 | 12600 | 0.000178571 |
| Kollicoat | 45000 | 0.00005 |
| PVA | 9500 | 0.000236842 |
| PVP K30 | 30000 | 0.000075 |
| HPMC | 10000 | 0.000225 |

TABLE 1B
List of 7 Surfactants initially screened

| Surfactant | MW | m/dm\hat{ }3 (22.5 mg/ml) |
|---|---|---|
| NDC | 414.55 | 0.005427572 |
| TPGS | 1000 | 0.00225 |
| AOT | 444.56 | 0.005061184 |
| Solutol HS | 344.53 | 0.006530636 |
| Tween 20 | 1227 | 0.001833741 |
| Tween 80 | 1300 | 0.001730769 |
| Hyamine | 448.08 | 0.005021425 |

### Screen Analysis

Immediately prior to analysis, samples were dispersed in a volume of water to give 1 mg/ml with respect to drug concentration. The z-average diameter (nm) of each of the solid drug nanodispersions was measured using dynamic light scattering (DLS; Malvern Zetasizer Nano ZS). Three measurements were made using automatic measurement optimisation and Malvern Zetasizer software version 7.11 for data analysis. The particles were considered hits if the below criteria were met.

### Nanodispersion Quality Assessment Criteria

A particle is determined a hit if it complies with the following criteria: candidates fully dispersed in water with no residual material, had a z-average diameter <1000 nm, standard deviation between each data set <15% and a polydispersity index <0.5.

Table 1C below lists the hits in terms of suitable hydrophilic polymers and surfactants.

TABLE 1C
hits of suitable hydrophilic polymers and surfactants (9 hits in all)

| Hydrophilic Polymer | Surfactant |
|---|---|
| PVA | Tween 20 |
| PVA | Tween 80 |
| PVA | NDC |
| PVA | AOT |
| Kollicoat | AOT |
| HPMC | AOT |
| PEG 1000 | AOT |
| PVP K30 | AOT |
| PVA | Solutol |

### Example MCV2—Preparing Higher Loading Maraviroc Solid Drug Nanodispersions

Aqueous stock solutions of polymer and surfactants were prepared at 22.5 mg ml⁻¹, maraviroc was prepared was at 70 mg ml⁻¹ in dichloromethane. An example 70 wt % drug loaded solid drug nanoparticle (SDN) was prepared as followed: Solutions were prepared at a 4:1 water:dichloromethane ratio, with 90 μl polymer, 45 μl surfactant and 265 μl water added to 100 μl maraviroc in dichloromethane. The resulting mixture was emulsified with a Covaris S2x for 30 seconds with a duty cycle of 20, intensity of 10 and 500 cycles/burst in frequency sweeping mode, after which samples were immediately cryogenically frozen. Samples were immediately sealed until analysis.

The polymer/surfactant combinations listed in Table 1C were subjected to serial modifications to incorporate higher maraviroc loading while reducing the polymer and surfactant components. The formulations were prepared as follows: 40 wt % maraviroc, 45 wt % polymer and 15 wt % surfactant; 50 wt % maraviroc, 40 wt % polymer and 10 wt % surfactant; 60 wt % maraviroc, 30 wt % polymer and 10 wt % surfactant; 70 wt % maraviroc, 20 wt % polymer and 10 wt % surfactant.

### Screen Analysis

Samples were analysed as for Example 1.

### Nanodispersion Quality Assessment Criteria

The quality of the nanodispersions was assessed as for Example 1.

TABLE 2A
Z-average and PDI of candidates at 30-70 wt % loading

| | | 30 wt % | | 40 wt % | | 50 wt % | | 60 wt % | | 70 wt % | |
|---|---|---|---|---|---|---|---|---|---|---|---|
| Maraviroc loading | | Z-Av/ | | Z-Av/ | | Z-Av/ | | Z-Av/ | | Z-Av/ | |
| Polymer | Surfactant | nm | PDI | nm | PDI | nm | PDI | nm | PDI | nm | PDI |
| PVA | Tween 20 | 517 | 0.30 | 815 | 0.60 | 855 | 0.38 | 1247 | 0.31 | 950 | 0.34 |
| PVA | Tween 80 | 623 | 0.29 | 122 | 0.41 | 957 | 0.48 | 1161 | 0.26 | 658 | 0.20 |
| PVA | NDC | 816 | 0.36 | 246 | 0.40 | 926 | 0.31 | 1750 | 0.31 | 823 | 0.17 |
| PVA | AOT | 643 | 0.35 | 724 | 0.36 | 1196 | 0.36 | 1205 | 0.26 | 728 | 0.30 |
| Kollicoat | AOT | 581 | 0.27 | 404 | 0.47 | 1124 | 0.56 | 1278 | 0.40 | 1550 | 0.71 |
| PEG1000 | AOT | 612 | 0.25 | 620 | 0.55 | 507 | 0.46 | 608 | 0.37 | 815 | 0.58 |
| HPMC | AOT | 913 | 0.14 | 747 | 0.43 | 931 | 0.31 | 1150 | 0.45 | 1824 | 0.38 |
| PVP K30 | AOT | 523 | 0.35 | 840 | 0.84 | 908 | 0.58 | 1951 | 0.46 | 3600 | 0.30 |
| PVA | Solutol | 538 | 0.34 | 494 | 0.39 | 1193 | 0.47 | 1392 | 0.30 | 1416 | 0.35 |

Three combinations of polymer and surfactant were found to be effective at 70 wt % loadings of maraviroc: PVA and Tween 80 (^{MVC}SDN_{PVA/TW80}); PVA and NDC (^{MVC}SDN_{PVA/NDC}); and PVA and AOT (^{MVC}SDN_{PVA/AOT}). The Z-average diameter, polydispersity index and zeta potential of the three successful 70 wt % maraviroc formulations is listed in Table 2B.

TABLE 2B
Z-average, PDI and zeta potential of nanodispersions formed
from the three successful 70 wt % maraviroc formulations

| | | Polydispersity | Zeta |
|---|---|---|---|
| Formulation | Z-Average/nm | Index | Potential/mV |
| ^{MVC}SDN_{PVA/TW80} | 658 | 0.2 | −12.8 |
| ^{MVC}SDN_{PVA/NDC} | 823 | 0.17 | −16.6 |
| ^{MVC}SDN_{PVA/AOT} | 728 | 0.3 | −25.3 |

### Example 3—In Vitro Permeation Studies of Maraviroc SDNs

### Cell Culture and Maintenance

Caco-2 cells were maintained in Dulbecco's Modified Eagle's Medium (DMEM) supplemented with 15% fetal bovine serum (FBS) (Gibco, UK). Cells were incubated at 37° C., 5% CO₂. Caco-2 cells were sub-cultured once −85% confluent. Cell counting and viability assessments were determined using propidium iodide exclusion on a NucleoCounter (Denmark).

### Transcellular Permeability of Maraviroc SDNs Across Caco-2 Monolayers

Transwells were seeded with 1.5×10⁵ cells per well and propagated to a monolayer over 21-days. During propagation, the media was aspirated from both apical and basolateral compartments and replaced with an equal volume of fresh pre-warmed (37° C.) media every other day, yielding transepithelial electrical resistance (TEER) values of >1000Ω. After 21-days, the media was aspirated, wells washed with pre-warmed (37° C.) HBSS and replaced with either DMSO dissolved maraviroc (<0.5% DMSO) or maraviroc nanodispersions, spiked into transport buffer, to a final concentration of 10 μM maraviroc with a specific activity of 25 μCi/mg [³H]-maraviroc. The suspensions were added to either apical or basolateral compartments and transport buffer added to the opposing chamber to quantify transport in both apical-to-basolateral (A>B) and basolateral-to-apical (B>A) directions. One-hundred microliters was sampled hourly from the opposing acceptor chamber over 4 h and replaced with an equal volume of fresh pre-warmed (37° C.) transport buffer. Collected samples were placed into empty 5 ml scintillation vials before mixing with liquid scintillation fluid (4 ml). Radioactivity was determined as disintegrations per minute (DPM) using a Packard Tri-carb 3100TR liquid scintillation counter. Apparent permeability (Pₐₚₚ) was determined by the amount of MVC transported over time using the equation below:

$$Papp = \frac{( dQ \text{/} dt ) \times v}{A \times {C}_{0}}$$

Where (dQ/dt) is the amount per time; v is the volume of the receiver compartment; A is the surface area of the filter; and Co is the starting concentration of the donor chamber. Apparent oral absorption was calculated using the Pₐₚₚ values: (A>B)/(B>A).

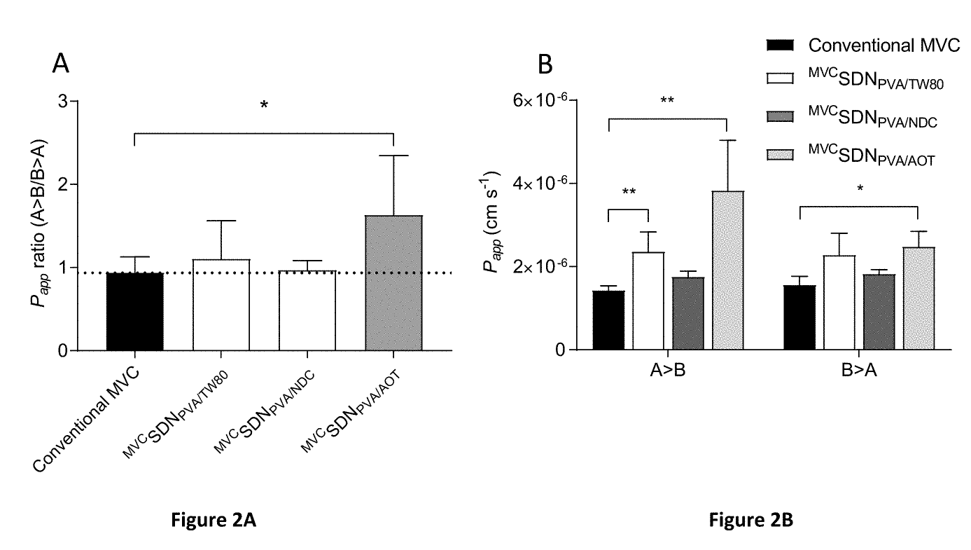

The three lead solid drug nanoparticle (SDN) formulations, namely ^{MVC}SDN_{PVA/TW80}, ^{MVC}SDN_{PVA/NDC} and ^{MVC}SDN_{PVA/AOT}, were prepared at 70 wt % maraviroc using tritium-labelled maraviroc ([³H]-maraviroc). Transcellular permeation of the aqueous maraviroc SDN nanodispersions was evaluated for both apical to basolateral and basolateral to apical permeability and compared to a conventional [³H]-maraviroc preparation (<0.5% aqueous DMSO). The results outlined in FIG. 2A indicate improved apparent oral absorption ((Pₐₚₚ(A>B)/Pₐₚₚ(B>A)) of two of the nanodispersed [³H]-maraviroc formulations (^{MVC}SDN_{PVA/TW80} and ^{MVC}SDN_{PVA/AOT}) when compared to a conventional [³H]-maraviroc preparation; >8% and >74% increase after 1 h incubation was observed, respectively. The mechanisms that underpin these observations are not fully understood; however, while the inventors do not wish to be bound by any particular theorem, various processes have been described which may account for the observed improvement, including paracellular permeation of intact particles, endocytosis of intact particles or indirect mechanisms which enable enhanced permeation of the dissolved drug.

^{MVC}SDN_{PVA/AOT} was found to have the highest Pₐₚₚ, which was significantly higher than conventional maraviroc formulations.

The integrity of the Caco-2 monolayers was investigated using the hydrophilic low permeability marker [¹⁴C]-mannitol following 1 h incubation with the conventional and each [³H]-maraviroc SDN candidate. Transport buffer was aspirated and the wells washed twice with pre-warmed (37° C.) HBSS. Subsequently, 0.1 ml of transport buffer containing [¹⁴C]-mannitol (50 μM, 2 μCi/ml) was added to the apical compartment of the test and control wells and 0.55 ml of transport buffer was added to the basolateral compartments. The plates were incubated at 37° C., 5% CO₂ for 1 h. Following incubation, 0.1 ml of the basolateral contents were sampled and placed into empty 5 ml scintillation vials before mixing with liquid scintillation fluid (4 ml). Radioactivity was determined as described above.

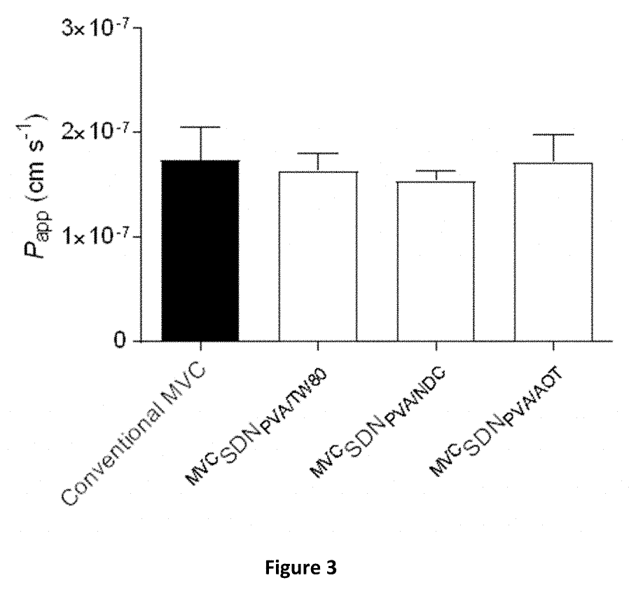

The results outlined in FIG. 3 highlight [¹⁴C]-mannitol Pₐₚₚ values of less than 0.953×10⁻⁶ cm s⁻¹ indicating the Caco-2 monolayers remained intact following exposure to each treatment.

### Example 4—In Vivo Oral Bioavailabilty and Tissue Distribution of Maraviroc SDNs

### In Vivo Rat Study

All animal work was conducted in accordance with the Animals (Scientific Procedures) Act 1986 (ASPA) implemented by the UK Home Office. The rodents were housed with environmental enrichment and a 12 h light/dark cycle at 21° C.±2° C. Free access to food and water was provided at all times. Following 7-days acclimatisation, adult male Wistar rats (280-330 g) were dosed with 10 mg Kg⁻¹ maraviroc at 10 μCi/mg, either as a conventional [³H]-maraviroc preparation (<5% DMSO) or as a [³H]-^{MVC}SDN_{PVA/AOT} nanodispersion (maraviroc solid drug nanoparticle (SDN) formulation containing PVA and AOT), using a 7-cm curved gavage needle. Subsequently, blood samples were collected (0.3 ml) at 0.5, 1.0, 1.5, 2.0 and 3.0 h post-dosing from the tail vein. At 4.0 h, the rats were sacrificed using cardiac puncture under terminal anaesthesia (isoflurane/oxygen), followed by immediate exsanguination of blood from the heart. Subsequently, an overdose of sodium pentobarbitone was administered using the same in situ puncture needle. Terminal tissue samples were collected, rinsed in PBS and dried on tissue before storing at −20° C. Blood samples were collected in heparinised Eppendorf tubes and centrifuged at 3,000 rpm for 5 min. The plasma layer was collected and stored at −20° C. prior to analysis.

### Quantification of Radiolabelled Plasma and Tissues

Plasma samples (0.1 ml) were transferred to scintillation vials before adding scintillation fluid (4 ml) (Meridian Biotechnologies, UK) and scintillation counting using a Packard Tri-carb 3100TR. Each dissected tissue was weighed individually and approximately 100 mg was placed into 20 ml scintillation vials. Tissue samples were submerged in 1 ml Soluene-350 (PerkinElmer, US) and incubated in a water bath at 50° C. for 18 h. After allowing to cool to room temperature, 0.2 ml of a 30% hydrogen peroxide solution was added to the dissolved sample and incubated for 60 min at room temperature. Subsequently, 0.09 ml of glacial acetic acid was added to each sample and incubated for a further 15 min at 50° C. Scintillation fluid (12 ml) was added to each sample and mixed via inversion. Scintillation counting was carried out using a Packard Tri-carb 3100TR.

### Statistical Analysis

Statistical analysis was performed using GraphPad Prism v.7 (US). Where statistical analysis is described, data normality was assessed with the Shapiro-Wilk test using StatsDirect v.3 (UK). Data were found to be normally distributed and unpaired, two-tailed t-tests were applied. Differences were considered statistically significant at *, P<0.05. Results are expressed as means and associated standard deviations. The pharmacokinetic parameters; maximum concentration (Cₘₐₓ), the time to Cₘₐₓ (Tₘₐₓ), trough concentrations (Cₘᵢₙ) and the average concentration (C_{avg}) were derived from the concentration-time profiles. The area under the curve, (AUC₀₋₄) and half-life (t %) were calculated using PKSolver.

### Bioavailability of Maraviroc Following Oral Administration of Maraviroc SDNs

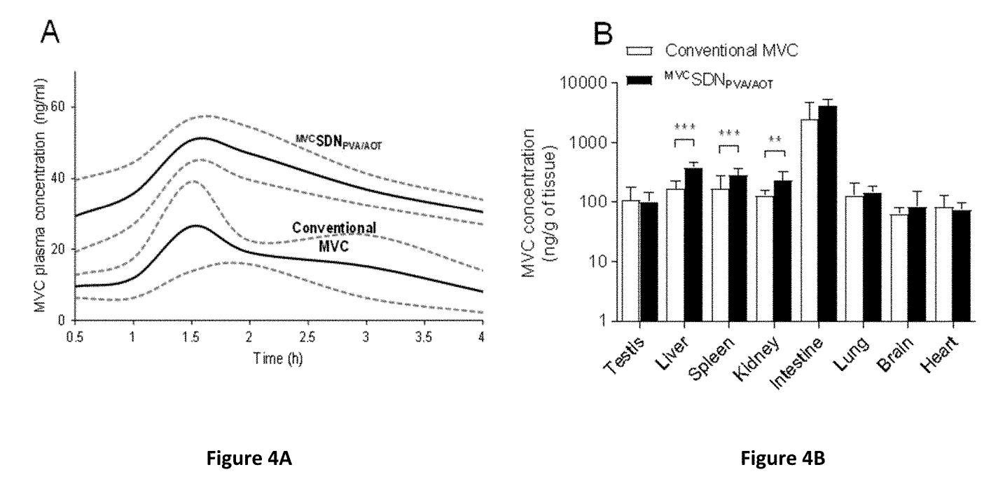

Each treatment group was orally-dosed at 10 mg Kg⁻¹ [3H]-maraviroc and blood samples were collected over 4 h to assess the different pharmacokinetic profiles. The results shown in FIG. 4A display increased Cₘₐₓ (50.74 vs. 26.52 ng ml⁻¹), increased Cₘᵢₙ (25.83 vs. 8.16 ng ml⁻¹), increased AUC (145.33 vs. 58.71 ng·h ml⁻¹), increased C_{avg} (38.38 vs. 15.17 ng ml⁻¹) an equivalent Tₘₐₓ (time to achieve Cₘₐₓ after dosing; 1.5 h), reduced Cₘₐₓ:Cₘᵢₙ ratio (1.96 vs. 3.25), and reduced Cₘₐₓ:C_{avg} ratio (1.32 vs. 1.75) for the ^{MVC}SDN_{PVA/AOT} dosed rats compared to the conventional solution in aqueous DMSO. The results highlight an increase in AUC and Cₘᵢₙ for ^{MVC}SDN_{PVA/AOT}, supporting a dose reduction strategy which ensures circulatory maraviroc concentrations remain efficacious, with an associated lower cost of therapy. The reduction in dose also has clear advantages for development of palatable and efficacious fixed does combinations. Maraviroc is generally well tolerated but Cₘₐₓ-driven postural hypotension has been described, and C_{avg} is an established parameter relating to efficacy. The favourable Cₘₐₓ:C_{avg} ratio reported here may enable development of a once-daily format, which maintains therapeutic exposure while avoiding the risk of concentration-dependent toxicities.

### Tissue Distribution of Maraviroc Following Oral Administration of Maraviroc SDNs

In addition to the enhanced pharmacokinetics, higher permeation was identified in most of the dissected tissues (FIG. 4B). In particular, statistically significant increases in [³H]-maraviroc were identified in the liver, spleen and kidney with a 2.2 (375.01 vs. 167.84 ng g⁻¹; P=<0.001), 1.6 (282.68 vs. 167.36 ng g⁻¹; P=<0.001) and 1.8-fold (231.31 vs. 130.47 ng g⁻¹; P=0.0057) increase, respectively. All other dissected tissues, except the heart and testis, were also shown to have increased maraviroc concentrations.

### Example 5—In Vitro Evaluation of Release Rates Using Rapid Equilibrium Dialysis (RED)

The rate of maraviroc release from ^{MVC}SDN_{PVA/AOT} was assessed across a size selective (8 kDa MWCO) membrane using RED plates and inserts. Either transport buffer (TB—consisting of Hanks balanced salt solution, 25 mM HEPES and 0.1% Bovine Serum Albumin (BSA), pH 7.4) or Simulated Interstitial Fluid (SIF—consisting of; dH₂O, 3.5% BSA and 0.2% γ-globulin, pH 7.4) were spiked with either DMSO dissolved maraviroc (<5% DMSO) or ^{MVC}SDN_{PVA/AOT}. A total of 1 mg [³H]-maraviroc (2 μCi mg) was added to the donor compartments for both preparations in 0.2 ml dH₂O with an additional 0.3 ml of either TB or SIF added to the donor chambers. One-millilitre of either TB or SIF was subsequently added to the corresponding acceptor chambers. The RED plates were sealed using Parafilm to avoid evaporation and placed on an orbital shaker (Heidolph Rotomax 120; 100 rpm, 6 h, 37° C.). Acceptor contents were subsequently sampled (0.6 ml) at 0.5, 1, 2, 3, 4, 5 and 6 h and replaced with an equal volume of fresh pre-warmed (37° C.) SIF or TB. Collected samples (0.1 ml) were placed into empty 5 ml scintillation vials before mixing with liquid scintillation fluid (4 ml). Radioactivity was determined as disintegrations per minute (DPM) using a Packard Tri-carb 3100TR liquid scintillation counter. Data are expressed as the amount of [³H]-maraviroc released and diffused across the size selective membrane as a first-order release rate constant calculated over the 6 h incubation.

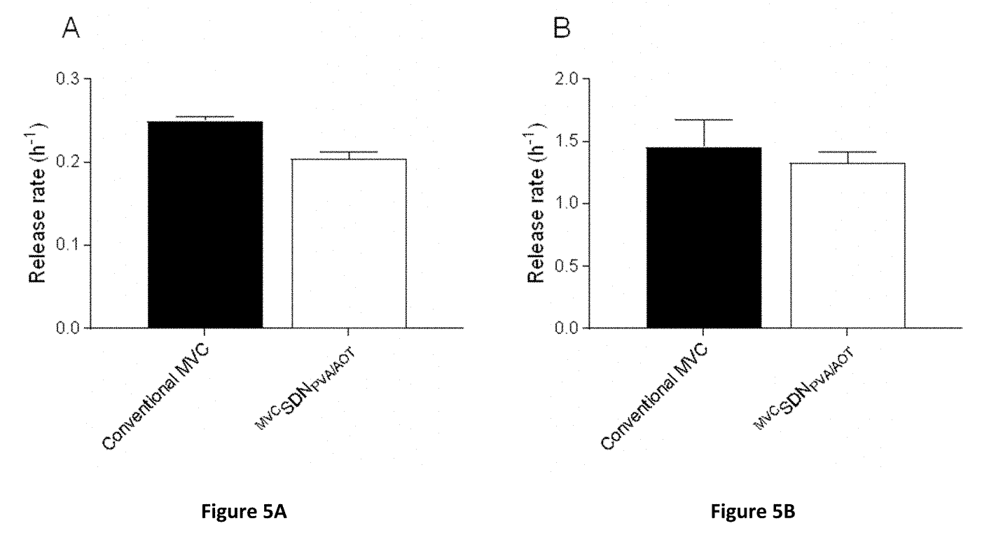

The first-order release rate constant results, outlined in FIG. 5, indicate a reduction in maraviroc release rate and subsequent diffusion across the size selective membrane when formulated as ^{MVC}SDN_{PVA/AOT} in both TB and SIF. Specifically, maraviroc release rate constant was shown to be 22.7% and 10% lower for ^{MVC}SDN_{PVA/AOT} compared to the release rate constant for the conventional preparation in TB and SIF, respectively. Interestingly, the overall rate of maraviroc release for both preparations was increased in SIF compared to TB which is possibly attributed to the higher protein content of the SIF buffer.

### Example 6—In Vivo Evaluation of Maraviroc SDNs as an Injectable

All animal work was conducted in accordance with the Animals (Scientific Procedures) Act 1986 (ASPA) implemented by the UK Home Office. The rodents were housed with environmental enrichment and a 12 h light/dark cycle at 21° C.±2° C. Free access to food and water was provided at all times. Following 7-days acclimatisation, adult male Wistar rats (280-330 g) were dosed intramuscularly with 10 mg/Kg⁻¹ maraviroc at 20 μCi/mg, after skin disinfection, with either a conventional [³H]-maraviroc preparation (<5% DMSO) or a [³H]-^{MVC}SDN_{PVA/AOT} nanodispersion into the left hind leg (musculus biceps femoris) using a 25G needle. Subsequently, blood samples were collected (0.25 ml) post-dosing from the tail vein until [³H]-maraviroc activity levels fell below the limits of detection (2 ng ml⁻¹). At the terminal timepoint, the rats were sacrificed using cardiac puncture under terminal anaesthesia (isoflurane/oxygen), followed by immediate exsanguination of blood from the heart. Subsequently, an overdose of sodium pentobarbitone was administered using the same in situ puncture needle.

### Quantification of Radiolabelled Plasma

Blood samples were collected in heparinised Eppendorf tubes and centrifuged at 3,000 rpm for 5 min. The plasma layer was collected and stored at −20° C. prior to analysis. Subsequently, 0.1 ml of each plasma sample was transferred into scintillation vials before adding scintillation fluid (4 ml) (Meridian Biotechnologies, UK) and scintillation counting using a Packard Tri-carb 3100TR.

### Statistical Analysis

Statistical analysis was performed using GraphPad Prism v.7 (US). Data normality was assessed with the Shapiro-Wilk test using StatsDirect v.3 (UK). Data were found to be normally distributed and unpaired, two-tailed t-tests were applied. For all comparisons, differences were considered statistically significant at *, P<0.05. Results are expressed as means and associated standard deviations. The pharmacokinetic parameters; maximum concentration (Cₘₐₓ), the time to Cₘₐₓ (Tₘₐₓ), trough concentrations (Cₘᵢₙ) and the average concentration (C_{avg}) were derived from the concentration-time profiles. The area under the curve, (AUC₀₋₄; AUC_{0-∞}) and terminal half-life (t %) were calculated using PKSolver.

In Vivo Study of ^{MVC}SDN_{PVA/AOT} Nanodispersion Administered by Intramuscular Injection

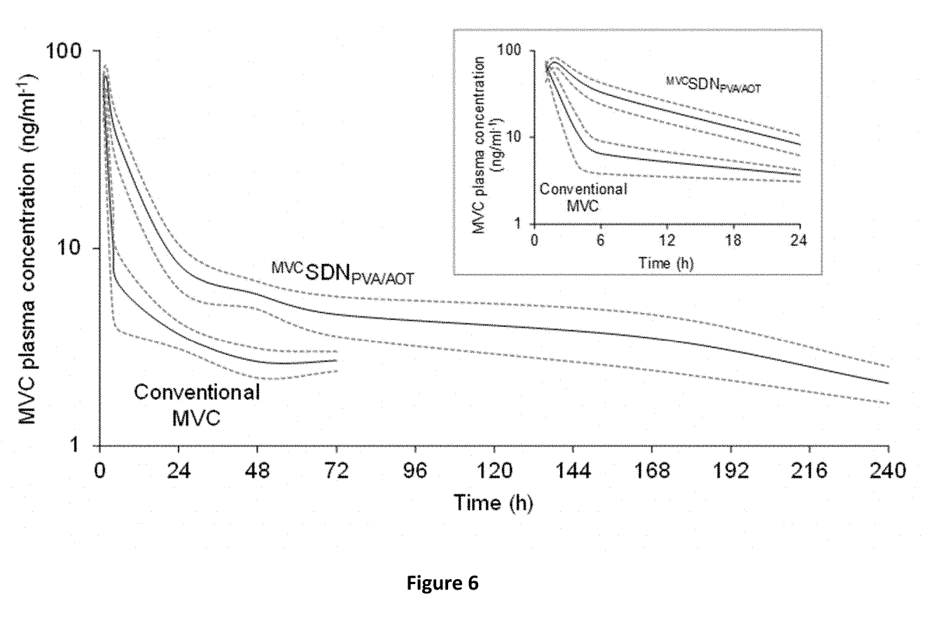

The results in FIG. 6 show both [³H]-maraviroc exposure over the initial 24 h (insert), encompassing the ‘burst event’ and exposure for the duration of the procedure, until [³H]-maraviroc plasma concentrations fell below the limits of quantification. The pharmacokinetic parameters are outlined in Table 3.

TABLE 3
The pharmacokinetic parameters of maraviroc following intramuscular
injection of [³H]-maraviroc (10 mg Kg⁻¹, 20 μCi mg [³H]-activity).

| Pharmacokinetic | Conventional | |
|---|---|---|
| parameter | MVC | ^{MVC}SDN_{PVA/AOT} |
| Cₘₐₓ (ng/ml⁻¹) | 71.67 | 72.96 |
| AUC_{t-∞} (ng · h/ml⁻¹) | 567.17 | 1959.71 |
| AUC₀₋₂₄ (ng · h/ml⁻¹) | 244.29 | 652.66 |
| Terminal half-life t½ (h) | 53.23 | 140.69 |
| Tₘₐₓ (h) | 1 | 2 |
| C₂₄ (ng/ml⁻¹) | 3.67 | 8.33 |
| C₄₈ (ng/ml⁻¹) | 2.69 | 5.85 |
| C₇₂ (ng/ml⁻¹) | 2.66 | 4.64 |
| C₁₆₈ (ng/ml⁻¹) | —* | 3.51 |
| C₂₄₀ (ng/ml⁻¹) | —* | 2.08 |

*Indicates that maraviroc was below the limits of detection

The data show a comparable Cₘₐₓ (72.96 vs 71.67 ng ml⁻¹), an increase in Tₘₐₓ (time to achieve Cₘₐₓ after dosing, 2.0 vs. 1.0 h), increased AUC₀₋₂₄ (652.66 vs. 244.29 ng·h ml⁻¹), increased AUC_{0-∞} (1959.71 vs. 567.17 ng·h ml⁻¹) and increased terminal half-life (t %) (140.69 vs. 53.23 h) for the nanodispersion dosed rats compared to the conventionally dosed rats. Following the initial rapid release of maraviroc, which led to the pronounced peak in plasma concentrations, the concentrations declined to 5.13% and 11.42% of the Cₘₐₓ value within 24 h for the conventional and ^{MVC}SDN_{PVA/AOT} preparations, respectively. After 24 h the [³H]-maraviroc plasma concentrations remained comparatively stable, declining steadily so that [³H]-maraviroc was detectable for 3- and 10-days post-dosing for the conventional and nanodispersion preparations, respectively. It is interesting to note that comparable maraviroc concentrations were observed at 1-week post-dosing (C₂₄₀) for ^{MVC}SDN_{PVA/AOT} and 1-day post-dosing (C₂₄) for the conventional maraviroc preparation (2.08 ng ml⁻¹ vs. 3.67 ng ml⁻¹). The terminal half-life (t %) for orally dosed maraviroc is −17 h compared to an observed (t %) of 53.23 h and 140.69 h for the intramuscularly dosed conventional maraviroc preparation and ^{MVC}SDN_{PVA/AOT}, respectively. Relatively low inter-individual variability was also noted for both treatment groups.

### Example 7—Screen for Suitable Excipient Combinations for the Production of Maraviroc Oil-Blended SDNs with Vitamin E as the Oil

Maraviroc was screened against 7 polymers and 6 surfactants, listed in Tables 4A and 4B below, in the presence of vitamin E. Each composition consisted of 50 wt % maraviroc, 8.33 wt % vitamin E, 31.67 wt % hydrophilic polymer and 10 wt % surfactant.

TABLE 4A
List of 7 hydrophilic polymers initially screened

| Polymer | MW | m/dm\hat{ }3 (22.5 mg/ml) |
|---|---|---|
| PEG 1000 | 1000 | 0.00225 |
| Pluronic F68 | 8400 | 0.000267857 |
| Pluronic F127 | 12600 | 0.000178571 |
| Kollicoat | 45000 | 0.00005 |
| PVA | 9500 | 0.000236842 |
| PVP K30 | 30000 | 0.000075 |
| HPMC | 10000 | 0.000225 |

TABLE 4B
List of 6 Surfactants initially screened

| Surfactant | MW | m/dm\hat{ }3 (22.5 mg/ml) |
|---|---|---|
| NDC | 414.55 | 0.005427572 |
| TPGS | 1000 | 0.00225 |
| AOT | 444.56 | 0.005061184 |
| Solutol HS | 344.53 | 0.006530636 |
| Tween 20 | 1227 | 0.001833741 |
| Tween 80 | 1300 | 0.001730769 |

The 50 wt % compositions were fabricated according to the following procedure: a stock solution containing 70 mg/ml maraviroc and Vitamin E combined (in a 6:1 weight ratio) was prepared in DCM. Polymers and surfactants were prepared in stock solutions of 22.5 mg/ml in water. To a small vial, 140.8 μL polymer, 44.4 μL surfactant and 231.5 μL water was added, followed by 83.3 μL of the drug solution. The resulting solution was sonicated for 15 seconds and immediately cryogenically frozen. Samples are then placed on a freeze dryer for 48 hours. Upon removal, the samples were immediately sealed before analysis by DLS.

Immediately prior to analysis, samples were dispersed in a volume of water to give 1 mg/ml with respect to drug concentration. The z-average diameter (nm) of each of the solid drug nanodispersions was measured using dynamic light scattering (DLS; Malvern Zetasizer Nano ZS). 3 measurements were made using automatic measurement optimisation and Malvern Zetasizer software version 7.11 for data analysis. The particles were considered hits if the below criteria were met.

### Nanodispersion Quality Assessment Criteria

A particle is determined a hit if it complies with the following criteria: candidates fully dispersed in water with no residual material, had a z-average diameter <1000 nm, standard deviation between each data set <5% and a polydispersity index <0.4.

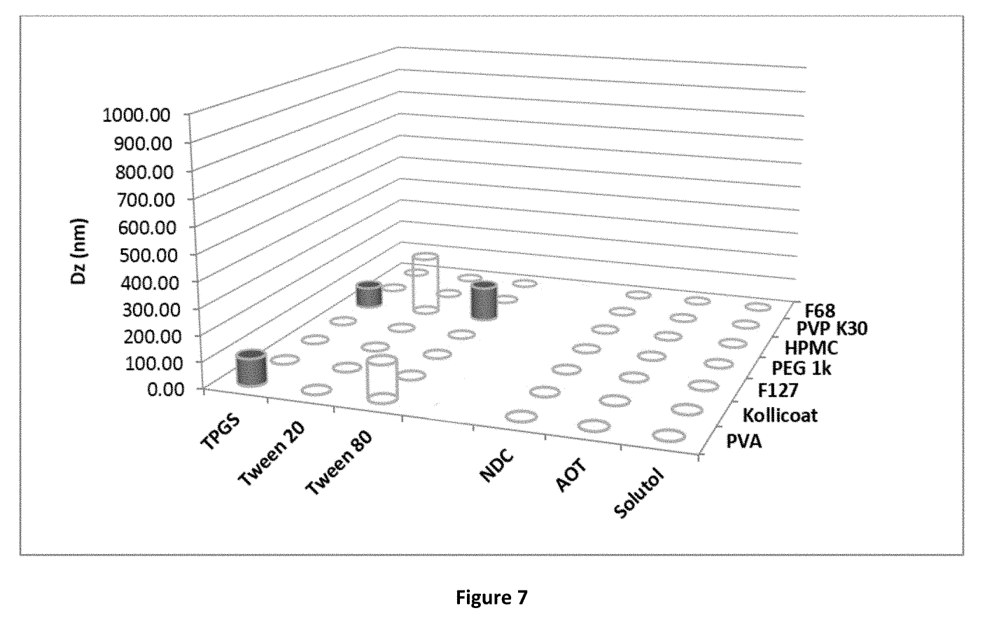

Table 4C lists the combinations of polymer and surfactant which were found to produce maraviroc oil-blended compositions which formed good nanodispersions of maraviroc when dispersed in aqueous solution, along with their DLS data in triplicate. This data is also represented in graphical form in FIG. 7.

TABLE 4C
DLS data for good nanodispersions formed by dispersion
of maraviroc oil-blended SDNs in water (50 wt
% maraviroc and 8.33 wt % Vitamin E)

| Formulation | | | | | |
|---|---|---|---|---|---|
| (polymer/surfactant) | Repeat | Dz (nm) | σ | PdI | Zeta (mV) |
| PVA/TPGS | 1 | 110 | 1 | 0.263 | −6.79 |
| | 2 | 110 | 1.5 | 0.266 | −18.0 |
| | 3 | 125 | 2.5 | 0.279 | −19.7 |
| HPMC/TPGS | 1 | 85 | 1 | 0.260 | −16.8 |
| | 2 | 90 | 0.5 | 0.270 | −14.9 |
| | 3 | 95 | 1 | 0.268 | −15.5 |
| HPMC/Tween80 | 1 | 170 | 1.5 | 0.280 | −32.0 |
| | 2 | 170 | 2.5 | 0.290 | −34.8 |
| | 3 | 175 | 1 | 0.296 | −33.5 |

In addition to showing that good nanodispersions were formed by the listed formulations, the DLS data shows that this result is highly reproducible. It should be noted that the combinations of polymer and surfactant in formulations which form good nanodispersions are different for oil-blended SDNs and conventional SDNs (see Example 1).

### Example 8—Release Rates of Maraviroc from Maraviroc Oil-Blended SDNs with Vitamin E as the Oil, as Determined by Rapid Equilibrium Dialysis (RED)

The rate of maraviroc release from the three maraviroc oil-blended SDN formulations found to most reliably and reproducibly produce nanodispersions in Example 7 was assessed across a size selective (8 kDa MWCO) membrane using RED plates and inserts. Control experiments were also run testing the rate of maraviroc release from aqueous maraviroc and a conventional maraviroc SDN (ACS_14-70 wt % maraviroc; 20 wt % PVA; and 10 wt % AOT as described in 2). Transport buffer (TB—consisting of Hanks balanced salt solution, 25 mM HEPES and 0.1% Bovine Serum Albumin (BSA), pH 7.4) was spiked with either DMSO dissolved maraviroc (<5% DMSO), a conventional maraviroc SDN or a maraviroc oil-blended SDN. A total of 1 mg maraviroc was added to the donor compartments for each preparation in 0.1 ml dH₂O with an additional 0.5 ml of TB added to the donor chambers. One-millilitre of TB was subsequently added to the corresponding acceptor chambers. The RED plates were sealed using Parafilm to avoid evaporation and placed on an orbital shaker (Heidolph Rotomax 120; 100 rpm, 6 h, 37° C.). Acceptor contents were subsequently sampled (0.6 ml) at 0.5, 1, 2, 3, 4, 5 and 6 h and replaced with an equal volume of fresh pre-warmed (37° C.) TB. Collected samples were analysed via HPLC.

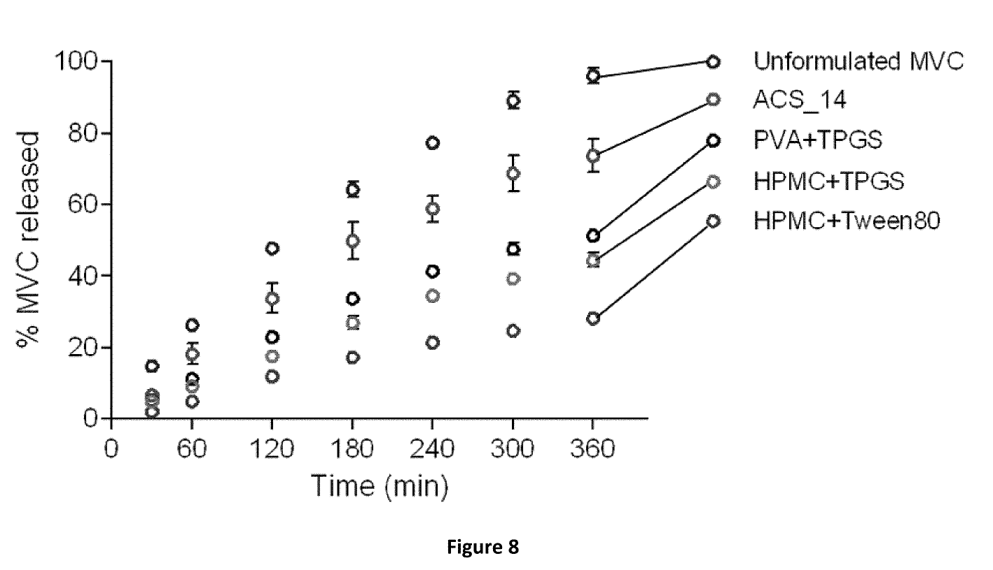

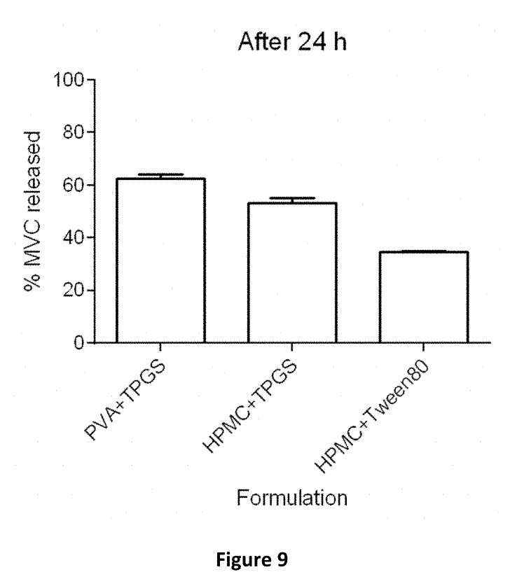

The rate of maraviroc released from each of the tested compositions is displayed in FIG. 8 and listed in Table 5. From this data it is clear to see that both the conventional and oil-blended SDNs release maraviroc at a slower rate than the aqueous maraviroc. It is also clear that the oil-blended SDNs release maraviroc at a slower rate than the conventional SDN. From this is can be concluded that the inclusion of an oil in the SDN formulation contributes to a slowing of the release of the water-insoluble active and can therefore be expected to contribute to a change in the pharmacokinetics of the formulation.

TABLE 5
The rate of release for aqueous maraviroc (unformulated
MVC), conventional maraviroc SDN (ACS_14)
and maraviroc oil-blended SDNs

| Treatment | Rate (h) |
|---|---|
| Unformulated MVC | 0.2491 |
| ACS_14 | 0.2295 |
| PVA + TPGS | 0.1433 |
| HPMC + TPGS | 0.1228 |
| HPMC + Tween80 | 0.0798 |

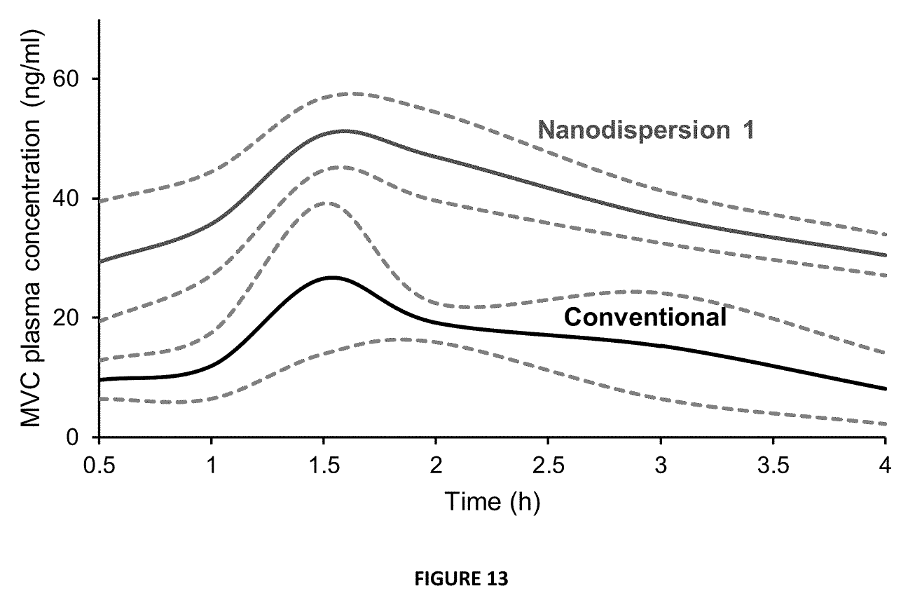

A comparison of the quantity of maraviroc released from the oil-blended SDNs (expressed as a percentage of the total maravoric content) after 24 hours is also included in FIG. 13 and demonstrates that, of the three maraviroc oil-blended SDNs tested, the formulation with HPMC and Tween 80 had the slowest release rate, while the composition with PVA and TPGS had the highest release rate.

### Example 9—Screen for Suitable Excipient Combinations for the Production of Maraviroc Oil-Blended SDNs with Soybean Oil as the Oil with a Maraviroc Loading of 50 wt %

Maraviroc was screened against the same 7 polymers and 6 surfactants as were used in Example 7, listed in Tables 4A and 4B, in the presence of soybean oil. Each composition consisted of 50 wt % maraviroc, 8.33 wt % soybean oil, 31.67 wt % hydrophilic polymer and 10 wt % surfactant.

Fabrication of Maraviroc Oil-Blended SDNs with Soybean Oil and Assessment of Nanodispersions Produced Therefrom

The 50 wt % compositions were fabricated according to the method described in Example 7, only substituting the Vitamin E used in Example 7 for soybean oil. These maraviroc oil-blended SDNs were then dispersed in water and analysed by DLS as described in Example 7.

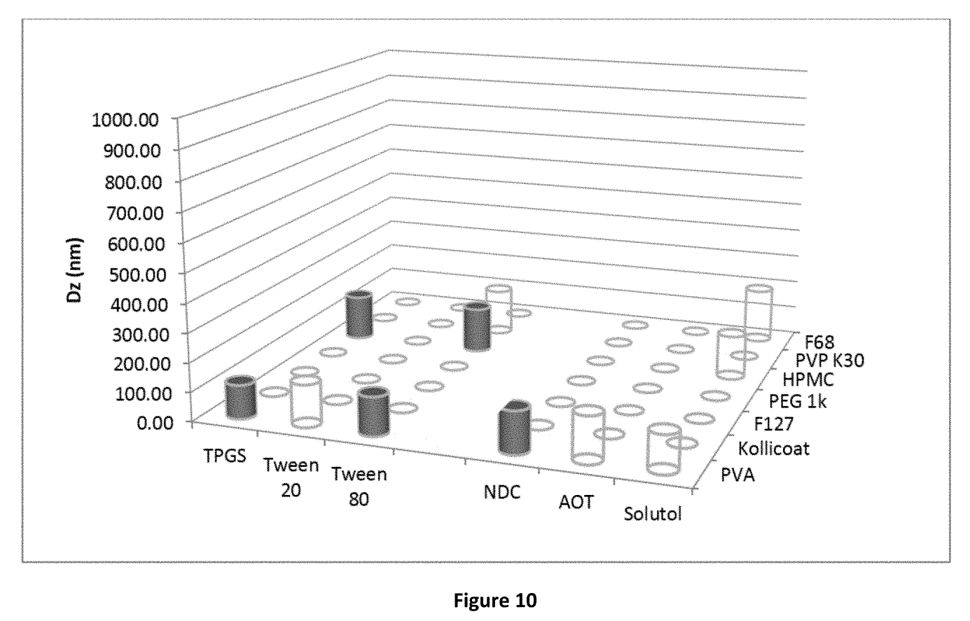

Table 6 lists the combinations of polymer and surfactant which were found to produce maraviroc oil-blended compositions which formed good nanodispersions of maraviroc when dispersed in aqueous solution, along with their DLS data in triplicate. This data is also represented in graphical form in FIG. 10.

TABLE 6
DLS data for good nanodispersions formed by dispersion
of maraviroc oil-blended SDNs in water (50 wt
% maraviroc and 8.33 wt % soybean oil)

| Formulation | | | | |
|---|---|---|---|---|
| (polymer/surfactant) | Repeat | Dz (nm) | PdI | Zeta (mV) |
| PVA/TPGS | 1 | 125 | 0.214 | −13.0 |
| | 2 | 130 | 0.145 | −15.3 |
| | 3 | 130 | 0.166 | −14.7 |
| HPMC/TPGS | 1 | 160 | 0.198 | −15.0 |
| | 2 | 175 | 0.160 | −25.8 |
| | 3 | 160 | 0.214 | −15.9 |
| PVA/Tween 80 | 1 | 145 | 0.166 | −27.4 |
| | 2 | 140 | 0.157 | −32.7 |
| | 3 | 140 | 0.179 | −26.3 |
| HPMC/Tween80 | 1 | 165 | 0.175 | −31.1 |
| | 2 | 160 | 0.126 | −27.4 |
| | 3 | 160 | 0.184 | −37.3 |
| PVA/NDC | 1 | 155 | 0.195 | −24.3 |
| | 2 | 160 | 0.173 | −21.3 |
| | 3 | 160 | 0.186 | −20.7 |

All five of the above formulations were found to be capable of forming good nanodispersions of maraviroc in aqueous media. It was found that maraviroc oil-blended SDNs with the following combinations of hydrophilic polymer and surfactant formed good nanodispersions in the most reliable and reproducible manner: PVA and TPGS; HPMC and TPGS; and PVA and NDC.

### Example 10—Screen for Suitable Excipient Combinations for the Production of Maraviroc Oil-Blended SDNs with Vitamin E as the Oil with a Maraviroc Loading of 60 Wt %

The three formulations which resulted in the most reproducible maraviroc oil-blended SDNs discovered in Example 9 were used to produce oil-blended SDNs with an increased maraviroc loading of 60 wt %. Each composition consisted of 60 wt % maraviroc, 10 wt % soybean oil, 20 wt % hydrophilic polymer and 10 wt % surfactant.

The 60 wt % compositions were fabricated according to the following procedure: a stock solution containing 70 mg/ml maraviroc and soybean oil combined (in a 6:1 weight ratio) was prepared in DCM. Polymers and surfactants were prepared in stock solutions of 22.5 mg/ml in water. To a small vial, 88.9 μL polymer, 44.4 μL surfactant and 266.7 μL water was added, followed by 100 μL of the drug solution. The resulting solution was sonicated for 15 seconds and immediately cryogenically frozen. Samples are then placed on a freeze dryer for 48 hours. Upon removal, the samples were immediately sealed before analysis by DLS.

Each of the three compositions was then used to produce an aqueous nanodispersion, the quality of which was then assessed by DLS, both procedures carried out as described in Example 9.

Table 7 lists combinations of polymer and surfactant which were found to produce maraviroc oil-blended compositions which formed good nanodispersions of maraviroc when dispersed in aqueous solution, along with their DLS data in triplicate. This data is also represented in graphical form in FIG. 10.

TABLE 7
DLS data for good nanodispersions formed by dispersion
of maraviroc oil-blended SDNs in water (60 wt
% maraviroc and 10 wt % soybean oil)

| Formulation | | | | |
|---|---|---|---|---|
| (polymer/surfactant) | Repeat | Dz (nm) | PdI | Zeta (mV) |
| PVA/TPGS | 1 | 160 | 0.146 | −21.8 |
| | 2 | 145 | 0.153 | −22.7 |
| | 3 | 155 | 0.149 | −21.2 |
| HPMC/TPGS | 1 | 175 | 0.166 | −21.2 |
| | 2 | 170 | 0.168 | −22.2 |
| | 3 | 170 | 0.173 | −18.5 |
| PVA/NDC | 1 | 180 | 0.189 | −29.7 |
| | 2 | 185 | 0.196 | −29.4 |
| | 3 | 180 | 0.190 | −31.6 |

All five of the above formulations with a 60 wt % loading of maraviroc were found to be capable of forming good nanodispersions of maraviroc in aqueous media. It was found that maraviroc oil-blended SDNs with the following combinations of hydrophilic polymer and surfactant formed good nanodispersions in the most reliable and reproducible manner: PVA and TPGS; and HPMC and TPGS.

### Example 11—Screen for Suitable Excipient Combinations for the Production of Maraviroc Oil-Blended SDNs with Vitamin E as the Oil with a Maraviroc Loading of 70 Wt %

The two formulations which resulted in the most reproducible maraviroc oil-blended SDNs discovered in Example 10 were used to produce oil-blended SDNs with an increased maraviroc loading of 60 wt %. Each composition consisted of 70 wt % maraviroc, 11.67 wt % soybean oil, 8.33 wt % hydrophilic polymer and 10 wt % surfactant.

The 70 wt % compositions were fabricated according to the following procedure: a stock solution containing 70 mg/ml maraviroc and soybean oil combined (in a 6:1 weight ratio) was prepared in DCM. Polymers and surfactants were prepared in stock solutions of 22.5 mg/ml in water. To a small vial, 37 μL polymer, 44.4 μL surfactant and 301.9 μL water was added, followed by 116.7 μL of the drug solution. The resulting solution was sonicated for 15 seconds and immediately cryogenically frozen. Samples are then placed on a freeze dryer for 48 hours. Upon removal, the samples were immediately sealed before analysis by DLS.

Each of the three compositions was then used to produce an aqueous nanodispersion, the quality of which was then assessed by DLS, both procedures carried out as described in Example 9.

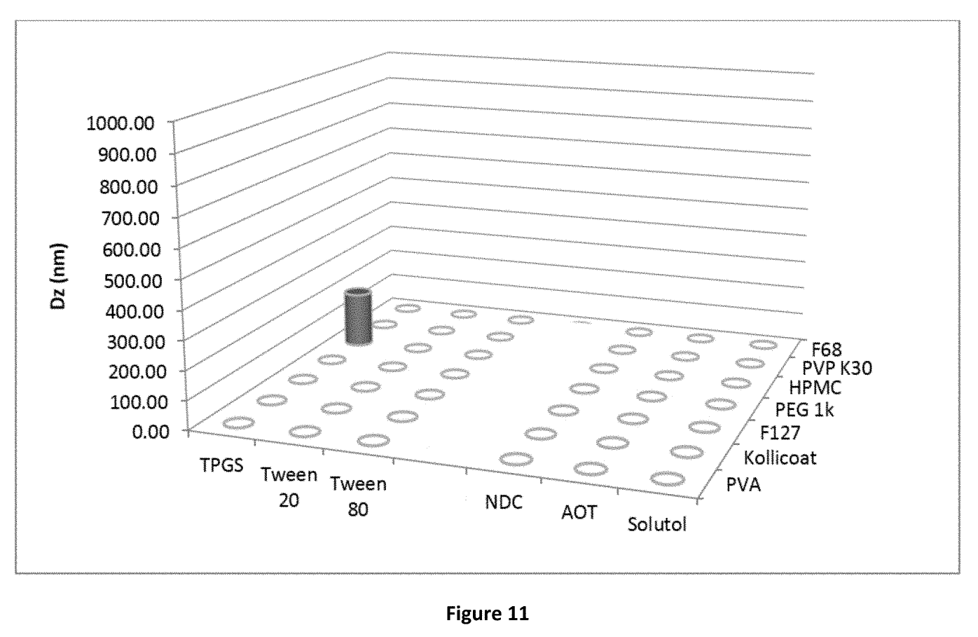

Table 8 lists the combinations of polymer and surfactant which were found to produce maraviroc oil-blended compositions which formed good nanodispersions of maraviroc when dispersed in aqueous solution, along with their DLS data in triplicate. This data is also represented in graphical form in FIG. 11.

TABLE 8
DLS data for good nanodispersions formed by dispersion
of maraviroc oil-blended SDNs in water (70 wt %
maraviroc and 11.67 wt % soybean oil)

| Formulation | Repeat | Dz (nm) | PdI | Zeta (mV) |
|---|---|---|---|---|
| HPMC/TPGS | 1 | 190 | 0.161 | −22.4 |
| | 2 | 185 | 0.158 | −20.6 |
| | 3 | 185 | 0.145 | −19.9 |

The formulation with HPMC and TPGS was found to form a good nanodispersion of maraviroc in aqueous media at a 70 wt % loading of maraviroc. Maraviroc oil-blended SDNs with this combination of hydrophilic polymer and surfactant were also found to form good nanodispersions in the most reliable and reproducible manner.

### Example 12—In Vitro Permeation Studies of Conventional Maraviroc SDNs and Maraviroc Oil-Blended SDNs

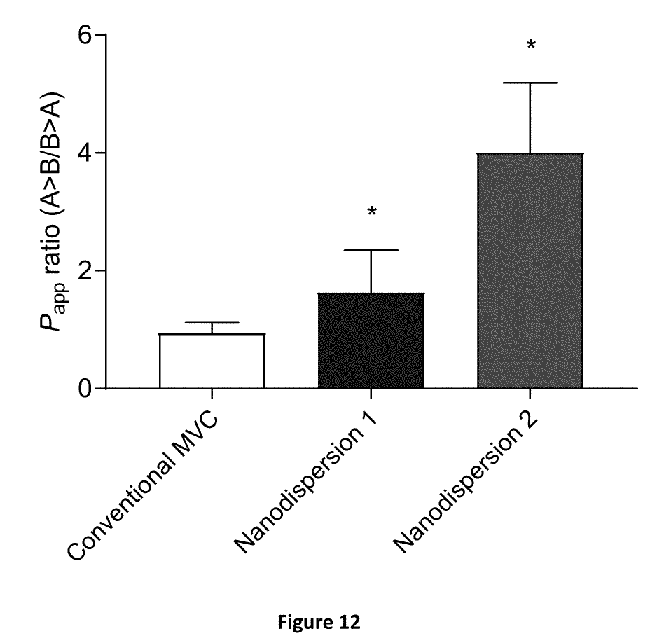

The permeation of a conventional maraviroc SDN (Nanodispersion 1, 70 wt % maraviroc; 20 wt % PVA; and 10 wt % AOT as described in 2), a maraviroc oil-blended SDN (Nanodispersion 2, 70 wt % maraviroc with soybean oil as described in Example 11) and aqueous maraviroc across a Caco-2 monolayer were measured as described in Example 8. The results of this experiment are displayed in FIG. 12.

From this experiment it was discovered that the maraviroc nanodispersions produced by both the conventional and oil-blended SDNs exhibited enhanced permeability over aqueous maraviroc. In addition, it was unexpectedly discovered that the enhancement provided by the oil-blended SDN (a 4.3-fold increase over the aqueous maraviroc) was greater than that of the conventional SDN (a 1.7-fold increase over the aqueous maraviroc).

### Example 13—Evaluation of In Vivo Pharmacokinetics for Orally Administered Conventional Maraviroc SDNs and Maraviroc Oil-Blended SDNs

All animal work was conducted in accordance with the Animals (Scientific Procedures) Act 1986 (ASPA) implemented by the UK Home Office. The rodents were housed with environmental enrichment and a 12 h light/dark cycle at 21° C.±2° C. Free access to food and water was provided at all times. Following 7-days acclimatisation, adult male Wistar rats (280-330 g) were dosed with 10 mg Kg⁻¹ maraviroc at 10 μCi/mg, as one of a conventional [³H]-maraviroc preparation (<5% DMSO), a [³H]-maraviroc conventional SDN (ACS_14-70 wt % maraviroc; 20 wt % PVA; and 10 wt % AOT as described in 2) nanodispersion or a [³H]-maraviroc oil-blended SDN nanodispersion (as described in 16) using a 7-cm curved gavage needle. Subsequently, blood samples were collected (0.3 ml) at 0.5, 1.0, 1.5, 2.0 and 3.0 h post-dosing from the tail vein. At 4.0 h, the rats were sacrificed using cardiac puncture under terminal anaesthesia (isoflurane/oxygen), followed by immediate exsanguination of blood from the heart. Subsequently, an overdose of sodium pentobarbitone was administered using the same in situ puncture needle. Terminal tissue samples were collected, rinsed in PBS and dried on tissue before storing at −20° C. Blood samples were collected in heparinised Eppendorf tubes and centrifuged at 3,000 rpm for 5 min. The plasma layer was collected and stored at −20° C. prior to analysis.

### Quantification of Radiolabelled Plasma and Tissues

Plasma samples (0.1 ml) were transferred to scintillation vials before adding scintillation fluid (4 ml) (Meridian Biotechnologies, UK) and scintillation counting using a Packard Tri-carb 3100TR. Each dissected tissue was weighed individually and approximately 100 mg was placed into 20 ml scintillation vials. Tissue samples were submerged in 1 ml Soluene-350 (PerkinElmer, US) and incubated in a water bath at 50° C. for 18 h. After allowing to cool to room temperature, 0.2 ml of a 30% hydrogen peroxide solution was added to the dissolved sample and incubated for 60 min at room temperature. Subsequently, 0.09 ml of glacial acetic acid was added to each sample and incubated for a further 15 min at 50° C. Scintillation fluid (12 ml) was added to each sample and mixed via inversion. Scintillation counting was carried out using a Packard Tri-carb 3100TR.

### Statistical Analysis

Statistical analysis was performed using GraphPad Prism v.7 (US). Where statistical analysis is described, data normality was assessed with the Shapiro-Wilk test using StatsDirect v.3 (UK). Data were found to be normally distributed and unpaired, two-tailed t-tests were applied. Differences were considered statistically significant at *, P<0.05. Results are expressed as means and associated standard deviations. The pharmacokinetic parameters; maximum concentration (Cₘₐₓ), the time to Cₘₐₓ (Tₘₐₓ), trough concentrations (Cₘᵢₙ) and the average concentration (C_{avg}) were derived from the concentration-time profiles. The area under the curve, (AUC₀₋₄) and half-life (t %) were calculated using PKSolver.

### Bioavailability of Maraviroc Following Oral Administration of a Maraviroc Oil-Blended SDN

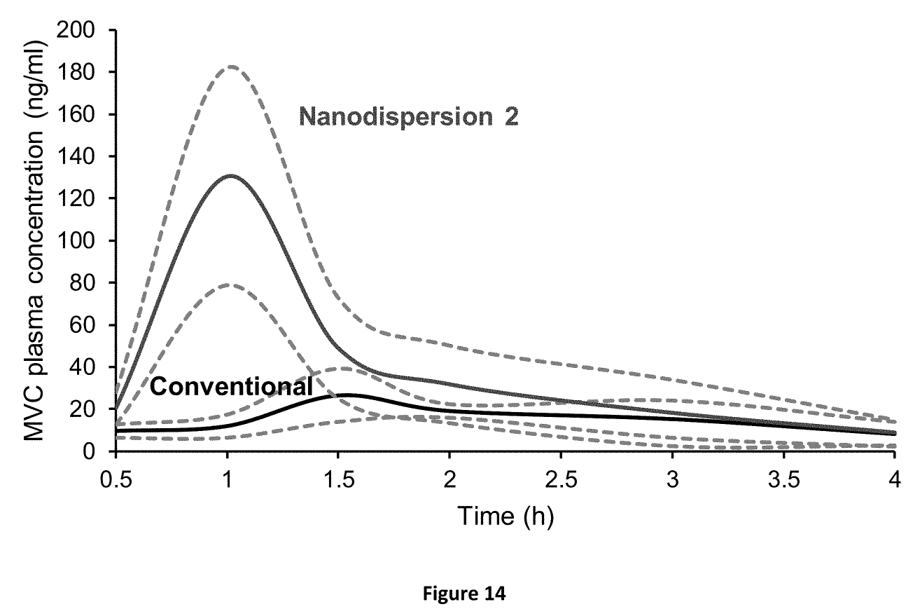

The plasma concentration of maraviroc at each time point was plotted on graph as exposure curves (FIGS. 13 and 14) and these curves were used to calculate various pharmacokinetic parameters for each of the three maraviroc formulations tested, which are tabulated in Table 9.

TABLE 9
Pharmacokinetic parameters of maraviroc following
oral dosing. Parameters were calculated from the
exposure curves outlined in FIGS. 13 and 14.

| | | Conventional | Maraviroc |
|---|---|---|---|
| | Aqueous | Maraviroc | Oil-blended |
| Pharmacokinetic parameter | maraviroc | SDN | SDN |
| Cₘₐₓ (ng ml⁻¹) | 26.52 | 50.74 | 130.31 |
| Cₘᵢₙ (ng ml⁻¹) | 8.16 | 25.83 | 8.88 |
| AUC₀₋₄ (ng · h ml⁻¹) | 58.71 | 145.33 | 146.24 |
| C_{avg} (ng ml⁻¹) | 15.17 | 38.38 | 43.06 |
| Tₘₐₓ (h) | 1.5 | 1.5 | 1.0 |
| Cₘₐₓ:Cₘᵢₙ ratio | 3.25 | 1.96 | 14.67 |

The aqueous nanodispersion of maraviroc produced using the conventional maraviroc SDN exhibited enhanced oral bioavailability over aqueous maraviroc. Unexpectedly, the maraviroc oil-blended SDN exhibited an even greater enhancement of the oral bioavailability.

### Tissue Distribution of Maraviroc Following Oral Administration of a Maraviroc Oil-Blended SDN

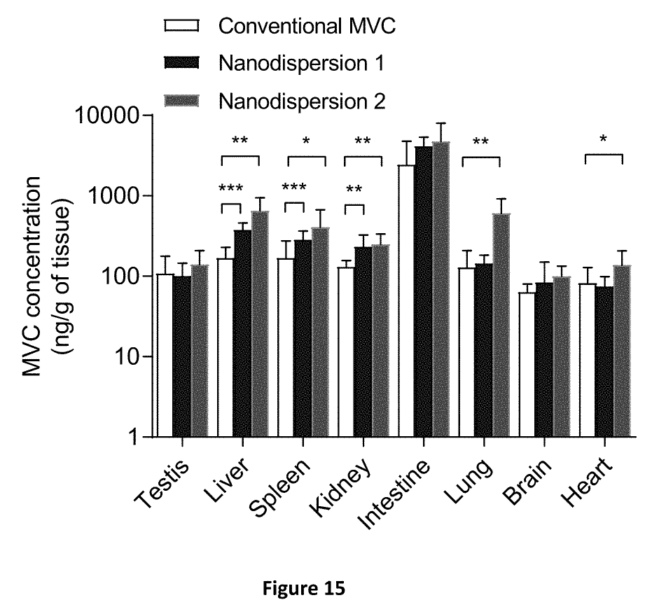

From analysis of the tissues, it was found that most tissues exhibited and increased maraviroc concentration for the conventional and oil-blended SDNs over the aqueous maraviroc (FIG. 15). The data is summarised in Table 10.

TABLE 10
The fold difference in maraviroc tissue concentrations for
conventional and oil-blended SDNs over aqueous maraviroc

| | Conventional Maraviroc SDN | | Maraviroc oil-blended SDN | |
|---|---|---|---|---|
| | Fold- | | Fold- | |
| | difference | | difference | |
| | (over | | (over | |
| Tissue | aqueous | Paired t-test | aqueous | Paired t-test |
| (n = 12) | maraviroc) | (two-tailed) | maraviroc) | (two-tailed) |
| Heart | 0.91 | Not significant | 1.69 | Not significant |
| Brain | 1.32 | Not significant | 1.58 | P = 0.0173 |
| Lung | 1.12 | Not significant | 4.69 | P = 0.0040 |
| Intestine | 1.70 | Not significant | 1.94 | Not significant |
| Kidney | 1.77 | P = 0.0057 | 1.91 | P = 0.0014 |
| Spleen | 1.69 | P = <0.0001 | 2.42 | P = 0.0227 |
| Liver | 2.23 | P = <0.0001 | 3.85 | P = 0.0010 |
| Testis | 0.93 | Not significant | 1.29 | Not significant |

Aqueous nanodispersions formed from both the conventional and oil-blended maraviroc SDNs demonstrated statistically significant increases in maraviroc tissue concentration in the kidney, spleen and liver. In addition, the maraviroc oil-blended SDN displayed statistically significant increases in both the lung and brain.

### Example 14—Release Rates of Maraviroc from Maraviroc Oil-Blended SDNs with Soybean Oil as the Oil, as Determined by Rapid Equilibrium Dialysis (RED)

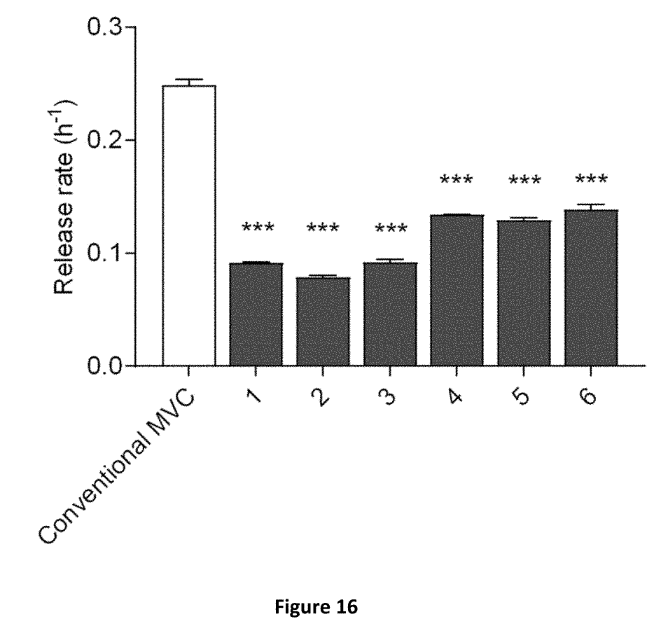

Rapid equilibrium dialysis was performed as per Example 8. The compositions tested and fold reduction in release rate compared to aqueous maraviroc are listed in Table 11. The compositions correspond to those found to form successful nanodispersions in Examples 9, 10 and 11. The data is also plotted as a bar graph in FIG. 16.

TABLE 11
Compositions of maraviroc oil-blended SDNs analysed
by RED and fold-reduction in maraviroc release rate

| | Composition | | Fold-decrease in |
|---|---|---|---|
| | (Maraviroc/ | | maraviroc release |
| | Soybean oil/ | | rate (as compared |
| Nanodispersion | polymer/ | Polymer and | to aqueous |
| # | surfactant) (wt %) | Surfactant used | maraviroc) |
| 1 | 50/8.33/31.67/10 | PVA and TPGS | 2.7 |
| 2 | 50/8.33/31.67/10 | HPMC and TPGS | 3.1 |
| 3 | 50/8.33/31.67/10 | PVA and NDC | 2.7 |
| 4 | 60/10/20/10 | HPMC and TPGS | 1.8 |
| 5 | 60/10/20/10 | PVA and TPGS | 1.9 |
| 6 | 70/11.67/8.33/10 | HPMC and TPGS | 1.8 |

As can be seen, there is a significant reduction in the rate of maraviroc release for each oil-blended SDN compared to aqueous maraviroc.

### Example 15—In Vivo Evaluation of Maraviroc Oil-Blended SDNs as a Long-Acting Injectable

All animal work was conducted in accordance with the Animals (Scientific Procedures) Act 1986 (ASPA) implemented by the UK Home Office. The rodents were housed with environmental enrichment and a 12 h light/dark cycle at 21° C.±2° C. Free access to food and water was provided at all times. Following 7-days acclimatisation, adult male Wistar rats (280-330 g) were dosed intramuscularly with 10 mg/Kg⁻¹ maraviroc at 20 μCi/mg, after skin disinfection, with either a conventional [³H]-maraviroc preparation (<5% DMSO) or a [³H]-maraviroc oil-blended SDN nanodispersion into the left hind leg (musculus biceps femoris) using a 25G needle. Subsequently, blood samples were collected (0.25 ml) post-dosing from the tail vein until [³H]-maraviroc activity levels fell below the limits of detection (2 ng ml⁻¹). At the terminal timepoint, the rats were sacrificed using cardiac puncture under terminal anaesthesia (isoflurane/oxygen), followed by immediate exsanguination of blood from the heart. Subsequently, an overdose of sodium pentobarbitone was administered using the same in situ puncture needle.

### Quantification of Radiolabelled Plasma

Blood samples were collected in heparinised Eppendorf tubes and centrifuged at 3,000 rpm for 5 min. The plasma layer was collected and stored at −20° C. prior to analysis. Subsequently, 0.1 ml of each plasma sample was transferred into scintillation vials before adding scintillation fluid (4 ml) (Meridian Biotechnologies, UK) and scintillation counting using a Packard Tri-carb 3100TR.

### Statistical Analysis

Statistical analysis was performed using GraphPad Prism v.7 (US). Data normality was assessed with the Shapiro-Wilk test using StatsDirect v.3 (UK). Data were found to be normally distributed and unpaired, two-tailed t-tests were applied. For all comparisons, differences were considered statistically significant at *, P<0.05. Results are expressed as means and associated standard deviations. The pharmacokinetic parameters; maximum concentration (Cₘₐₓ), the time to Cₘₐₓ (Tₘₐₓ), trough concentrations (Cₘᵢₙ) and the average concentration (C_{avg}) were derived from the concentration-time profiles. The area under the curve, (AUC₀₋₄; AUC_{0-∞}) and terminal half-life (t %) were calculated using PKSolver.

### In Vivo Study of Maraviroc Oil-Blended SDN Nanodispersions Administered by Intramuscular Injection

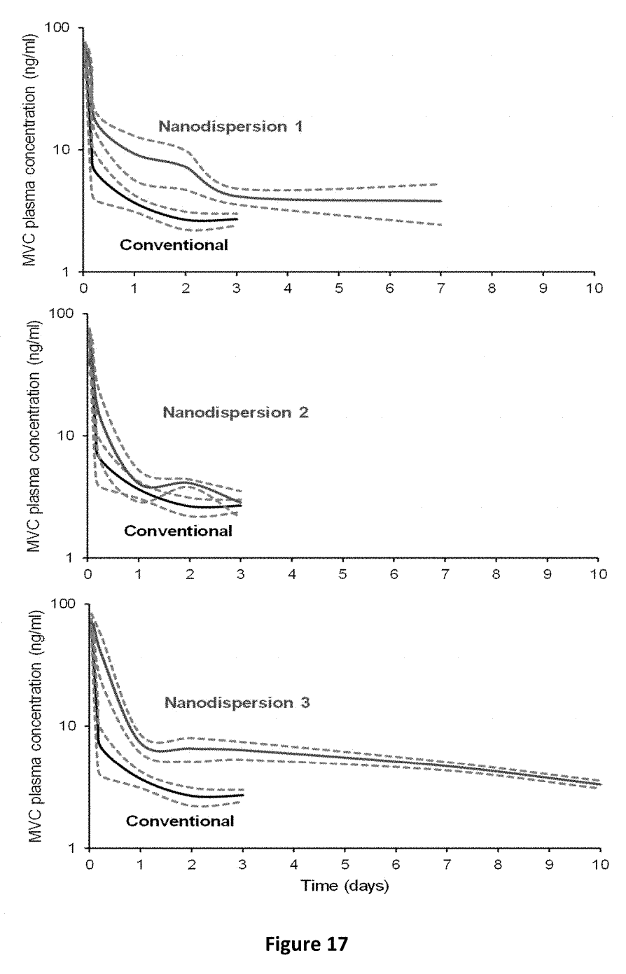

Nanodispersions 1 to 3 (as described in Example 14) were selected for in vivo study as potential long-acting injectables due to their having the slowest release rate of the formulations tested see FIG. 16 and Table 11). A control experiment using aqueous maraviroc (“Conventional maraviroc”) was also performed for comparison. The plasma concentration of maraviroc at each time point was plotted as exposure curves (FIG. 17) and these curves were used to calculate various pharmacokinetic parameters for each of the three maraviroc formulations tested, which are shown in Table 12.

TABLE 12
Pharmacokinetic parameters of maraviroc following intramuscular
injection. Parameters were calculated from the exposure curves
outlined in FIG. 17. The compositions of the maraviroc oil-
blended SDN used are described in Example 14

| Pharmacokinetic | Conventional | Nanodis- | Nanodis- | Nanodis- |
|---|---|---|---|---|
| parameter | maraviroc | persion 1 | persion 2 | persion 3 |
| Cₘₐₓ (ng ml⁻¹) | 71.67 | 62.88 | 50.58 | 69.85 |
| AUC_{0-∞} (ng · h ml⁻¹) | 567.17 | 1720.51 | 628.62 | 2821.3 |
| AUC₀₋₂₄ (ng · h ml⁻¹) | 244.29 | 472.19 | 356.76 | 714.85 |
| Terminal half-life | 53.23 | 121.44 | 33.19 | 196.04 |
| (t½) | | | | |
| C₂₄ (ng ml⁻¹) | 3.67 | 9.30 | 4.11 | 7.23 |
| C₄₈ (ng ml⁻¹) | 2.69 | 7.28 | 4.08 | 6.50 |
| C₇₂ (ng ml⁻¹) | 2.66 | 4.18 | 2.84 | 6.32 |
| C₁₆₈ (ng ml⁻¹) | —* | 3.81 | —* | 4.67 |
| C₂₄₀ (ng ml⁻¹) | —* | —* | —* | 3.30 |

The results show that maraviroc was still detectable in nanodispersions 1 and 3 at 7 and 10 days post-injection respectively. Conversely, the conventional (aqueous) maraviroc and nanodispersion 2 ceased to be detectable after 3 days. Nanodispersion 1 also displayed a 3-fold increase in AUC_{0-∞} and a 2.3-fold increase in t½ over the aqueous maraviroc, a significant improvement. Similarly, nanodispersion 3 displayed a 4.9-fold increase in AUC_{0-∞} and a 3.6-fold increase in t % over the aqueous maraviroc.

1. A solid maraviroc composition, comprising nanoparticles including maraviroc dispersed within a mixture of at least one hydrophilic polymer and at least one surfactant;
wherein the hydrophilic polymer is selected from polyvinyl alcohol (PVA), a polyvinyl alcohol-polyethylene glycol graft copolymer, polyethylene glycol, hydroxypropyl methyl cellulose (HPMC) and polyvinylpyrrolidone (PVP), or a combination thereof; and
the surfactant is selected from a polyoxyethylene sorbitan fatty acid ester, sodium deoxycholate, dioctyl sodium sulfosuccinate and polyethyleneglycol-12-hydroxystearate, polyvinyl alcohol (PVA) or a combination thereof.

2. A solid maraviroc composition according to claim 1, wherein the nanoparticles including maraviroc have an average particle size of less than or equal to 1 micron (μm).

3. A solid maraviroc composition according to claim 1, wherein the nanoparticles including maraviroc have an average particle size between 100 and 800 nm.

4. A solid maraviroc composition according to claim 1, wherein the polydispersity of the nanoparticles including maraviroc is less than or equal to 0.8.

5. A solid maraviroc composition according to claim 1, wherein the hydrophilic polymer is selected from polyvinyl alcohol (PVA), a polyvinyl alcohol-polyethylene glycol graft copolymer and hydroxypropyl methyl cellulose (HPMC), or a combination thereof.

6. A solid maraviroc composition according to claim 1, wherein the hydrophilic polymer is PVA.

7. A solid maraviroc composition according to claim 1, wherein the surfactant is selected from polysorbate 80, sodium deoxycholate, dioctyl sodium sulfosuccinate and polyethyleneglycol-12-hydroxystearate or a combination thereof.

8. A solid maraviroc composition according to claim 1, wherein the hydrophilic polymer is PVA and the surfactant is selected from polysorbate 80, sodium deoxycholate and dioctyl sodium sulfosuccinate.

9. A solid maraviroc composition according to claim 1 comprising:
60-80% maraviroc;
15-25 wt % PVA; and
5-15 wt % dioctyl sodium sulfosuccinate.

10. A pharmaceutical composition in a solid dosage form comprising a solid composition according to claim 1, and optionally one or more additional pharmaceutically acceptable excipients.

11. A process for preparing a solid composition according to claim 1, the process comprising:
(a) preparing an oil in water emulsion using a volatile oil comprising:
an oil phase comprising maraviroc; and
an aqueous phase comprising a hydrophilic polymer and a surfactant, each as defined in claim 1; and
(b) removing the volatile oil and water to form the solid composition.

12. A process for preparing a solid composition according to claim 1, the process comprising:
preparing a single phase solution comprising maraviroc, a hydrophilic polymer as defined in claim 1, and a surfactant as defined in claim 1, in one or more solvents; and
spray-drying the mixture to remove the one or more solvents to form the solid composition.

13. An aqueous dispersion, comprising a plurality of nanoparticles dispersed in an aqueous medium, the nanoparticles comprising maraviroc, at least one hydrophilic polymer and at least one surfactant;
wherein the hydrophilic polymer is selected from polyvinyl alcohol, a polyvinyl alcohol-polyethylene glycol graft copolymer, polyethylene glycol, hydroxypropyl methyl cellulose and polyvinylpyrrolidone, or a combination thereof; and
wherein the surfactant is selected from a polyoxyethylene sorbitan fatty acid ester, sodium deoxycholate, dioctyl sodium sulfosuccinate, and polyethyleneglycol-12-hydroxystearate, polyvinyl alcohol (PVA), or a combination thereof.

14. An aqueous dispersion according to claim 13, wherein the nanoparticles comprising maraviroc, the at least one hydrophilic polymer and the at least one surfactant have an average particle size of less than or equal to 1 micron (μm).

15. An aqueous dispersion according to claim 13, wherein the nanoparticles comprising maraviroc, the at least one hydrophilic polymer and the at least one surfactant have an average particle size between 100 and 800 nm.

16. An aqueous dispersion according to claim 13, wherein the average zeta potential of the nanoparticles comprising maraviroc, the at least one hydrophilic polymer and the at least one surfactant when dispersed in an aqueous medium is between −100 and +100 mV.

17. A process for preparing an aqueous dispersion according to claim 13, comprising dispersing a solid maraviroc composition in an aqueous medium, wherein the solid maraviroc composition comprises the nanoparticles comprising maraviroc dispersed within a mixture of the at least one hydrophilic polymer and the at least one surfactant.

18. A pharmaceutical composition comprising an aqueous dispersion as claimed in claim 13 and optionally one or more additional pharmaceutically acceptable excipients.

19. (canceled)

20. (canceled)

21. A method of treating and/or preventing a retroviral infection (e.g. HIV), the method comprising administering a therapeutically effective amount of a solid composition according to claim 1 to a patient suffering from or at risk of suffering from a retroviral infection.

22. The method according to claim 21 for the treatment or prevention of a retrovirus infection, such as HIV, wherein the solid composition administered in combination with one or more other antiretroviral agents.
